(19) United States
(12) Patent Application Publication
(10) Pub. No.: US 2011/0171591 A1
(43) Pub. Date: Jul. 14, 2011

(54) ORTHODONTIC COMPOSITION WITH HEAT MODIFIED MINERALS

(76) Inventors: David T. Amos, St. Paul, MN (US); Geoffrey P. Morris, White Bear Lake, MN (US)

(21) Appl. No.: 13/120,956

(22) PCT Filed: Sep. 8, 2009
(86) PCT No.: PCT/US09/56180

§ 371 Date: Mar. 25, 2011

Related U.S. Application Data
(60) Provisional application No. 61/101,339, filed on Sep. 30, 2008.

(51) Int. Cl. A61C 7/16; A61K 6/02; A61C 17/00

(52) U.S. Cl. 433/9; 106/35; 433/216

(57) ABSTRACT

The invention provides an orthodontic composition, related methods and packaged articles that include a hardenable component, a hardener, and a heat-modified inorganic mineral filler. The composition displays improved hardened remnant cleanup compared with compositions using traditional hard mineral fillers, while maintaining acceptable handling, bond strength and mechanical properties. Bond strength enhancement is achieved by heat-modifying a soft mineral filler, a process by which water of hydration is eliminated from the microstructure of the mineral to form a non-hydrated phase. By using heat-modified mineral fillers that are soft relative to human enamel, the hardened orthodontic composition can be conveniently removed using a lowspeed abrasive disk or other mild abrasion process that is safer and more comfortable for the patient. The composition is especially beneficial for use in bonding orthodontic appliances to teeth.

### BACKGROUND OF THE INVENTION

1. Field of the Invention

The present invention broadly pertains to compositions, methods and articles in the field of orthodontia. More particularly, this invention pertains to an orthodontic composition containing a heat-modified inorganic mineral filler, as well as related methods and packaged articles.

2. Description of the Related Art

Orthodontics is a specialized field of dentistry in which malpositioned teeth are diagnosed and guided into proper locations in the oral cavity. Orthodontic treatment is commonly used in correcting defects in a patient's bite (also called occlusion) along with promoting better hygiene and improving the overall aesthetics of the teeth.

Orthodontic treatment often involves the use of tiny slotted appliances known as brackets, which are generally affixed to the patient's anterior, cuspid, and bicuspid teeth. After the brackets have been placed on the teeth, an archwire is received into the slot of each bracket and applies steady, continuous forces to guide respective teeth to orthodontically correct positions. End sections of the archwire are typically received in anchoring appliances known as buccal tubes that are bonded to the patient's molar teeth. The combination of brackets, archwires, and buccal tubes is commonly referred to as an orthodontic brace, or “braces”.

It is common practice for orthodontists to use adhesives to bond orthodontic appliances to the teeth surfaces. In the direct bonding method, an adhesive is applied to the appliance base, the appliance is mounted onto a tooth, excess adhesive flash removed, and the adhesive finally hardened. Alternatively, an indirect method may be used, in which appliances are first adhesively bonded to a replica of the patient's teeth (commonly made from plaster or orthodontic stone) and a resilient transfer tray formed over the appliances and the replica. The transfer tray is then used to carefully detach the appliances from the replica and simultaneously re-bond the appliances to the patient's teeth, again using an appropriate adhesive. A suitable adhesive should exhibit a sufficiently high bond strength for maintaining adhesion of the appliance to the tooth surface over the duration of the treatment process, which can last two years or longer.

At the conclusion of orthodontic treatment, the appliances are debonded from the teeth of the patient. There are also situations, however, in which appliances are debonded from teeth prior to the conclusion of treatment. For example, the orthodontist may remove and re-position just one or two appliances in the middle of treatment to correct a malpositioned appliance or achieve a particular treatment goal. It is also possible that one or more appliances may become accidentally debonded when the patient bites down on a hard food substance.

In each of the above situations, adhesive remnant is usually left on the tooth surface where the appliance was debonded and this remnant needs to be meticulously removed by the treating professional. Conventional orthodontic adhesives are highly crosslinked and usually contain hard inorganic fillers, such as silica. Moreover, these fillers are often present in amounts exceeding 50 weight percent of the adhesive in order to achieve sufficiently high strength and acceptable adhesive handling properties. Once hardened, these materials tend to be difficult and time-consuming to remove from the patient's teeth. Today, remnant adhesive is commonly removed using a rotary hand tool using, for example, a fluted tungsten carbide burr operating in the range of 20,000 to 200,000 rpm. Because tungsten carbide is a relatively hard material compared with tooth enamel, the mere use of this hand tool incurs a risk that the tip will damage the enamel. Because of the associated risks, the task of removing adhesive remnant is generally only carried out by treating professionals that have extensive training and/or experience.

Since the grinding of adhesive from the tooth takes a substantial amount of time and attention, it is often considered a cumbersome procedure for treating professional. The grinding action is also generally regarded as uncomfortable for the patient. There are also other risks associated with using a high speed rotary hand tool on teeth. High rotation speeds cause frictional heating of the enamel surface by the fluted burr. Although this heating can be alleviated using a stream of coolant water, many clinicians avoid using such a coolant because it is often easier to see remnant adhesive on the enamel when the site is dry. Recent studies suggest that the temperature rise in the pulpal chamber caused by grinding using a fluted burr can be significant and that damage to the pulp can occur at these temperatures (see Jonke et al. World Journal of Orthodontics, Vol. 7, p. 357 (2006) and Zach et al. Oral Surg. Oral Med. Oral Pathol. (1965) Vol. 19, pp. 515-530).

Some references have suggested using lower amounts of filler in an orthodontic adhesive than the amounts typically used, to ease removal of residual adhesive. Some of these concepts are described in Gwinnett and Gorelick, Am. J. Orthod., Vol. 71, No. 6, pp. 651-665 (June 1977), Retief and Denys, “Finishing of Enamel Surfaces After Debonding of Orthodontic Attachments”, Angle Orthodontist, Vol. 49, No. 1, pp. 1-10 (January 1979), and U.S. Pat. No. 3,629,187 (Waller, et al.). However, removal of residual adhesive containing these fillers still can lead to enamel damage because hardened tools are typically used to grind away the residual adhesive. It remains a challenge to facilitate the removal of remnant adhesive while minimizing the risk of damaging the enamel and pulp of the tooth.

### SUMMARY OF THE INVENTION

The present invention provides an orthodontic composition and related methods displaying improved residual remnant cleanup compared with compositions using traditional hard mineral fillers, while maintaining acceptable handling, bond strength and mechanical properties. This advantage can be achieved by using a composition that includes a heat-modified mineral. Heat modification is a process by which water of hydration is eliminated from the microstructure of a mineral to form a non-hydrated phase. By using heat-modified mineral fillers that are soft relative to human enamel, the hardened orthodontic composition can be conveniently removed using a low-speed abrasive disk or other mild abrasion process that is both gentle and safe on the enamel.

In some cases, the process of heat modification has the additional effect of transforming the filler particles from a generally plate-shaped (or flat) configuration to a generally spherical configuration. It was determined that these heat-modified fillers provide a surprising increase in maximum filler loading compared with the unmodified virgin filler. Advantageously, this increase in maximum filler loading allows greater freedom to optimize the packing density of the filler in the composition. The packing density of a composite is known to be correlated with the composite's strength. This is further demonstrated by test measurements on these hardened compositions. Prior to this discovery, there was no indication that compositions using heat-modified fillers are advantageous over compositions that do not use heat-modified fillers. However, compositions using the heat-modified soft mineral particles are shown to yield a superior bond strength and diametral tensile strength when compared with those using the virgin mineral particles prior to heat-modification.

In one aspect, the present invention is directed to an orthodontic composition comprising a hardenable component, a hardener, and a heat-modified inorganic mineral filler.

In another aspect, the invention is directed to a method of making an orthodontic composition comprising providing a hardenable component, providing a hardener, providing a virgin inorganic mineral filler that includes a hydrated phase, heating the virgin inorganic mineral filler to a temperature sufficient to transform at least a portion of the hydrated phase to a non-hydrated phase in order to make a heat-modified filler, and combining the hardenable component, hardener, and heat-modified filler to form the orthodontic composition.

In another aspect, the invention is directed to a packaged article comprising an orthodontic appliance having a base for bonding the appliance to a tooth, a composition extending across the base of the appliance, wherein the composition comprises a hardenable component, a hardener, and a heat-modified inorganic mineral filler, and a container at least partially surrounding the orthodontic appliance and the composition.

In still another aspect, the invention is directed to a method for removing cured orthodontic composition from a tooth comprising providing a tooth surface having a cured orthodontic composition on at least a portion thereon, the composition comprising a hardenable component, a hardener, and a heat-modified inorganic filler, and applying an abrasive to the tooth surface to remove the composition from the tooth, wherein the abrasive has a Mohs hardness that is less than 5.

### BRIEF DESCRIPTION OF THE DRAWINGS

The present invention will be described with respect to the attached Figures, in which:

FIG. 1 is a perspective view looking at the base of an orthodontic appliance pre-coated with an exemplary composition that is contacted in part by a release substrate.

FIG. 2 is side cross-sectional view of a certain embodiment of the present invention illustrating a packaged article including an orthodontic appliance coated with an exemplary composition thereof in a container with a removable cover.

FIG. 3a is a thermal gravimetric-differential thermal analysis trace for virgin chlorite filler.

FIG. 3b is a thermal gravimetric-differential thermal analysis trace for a chlorite filler that has been heat modified by calcining at 800° C.

FIG. 3c is a thermal gravimetric-differential thermal analysis trace for a chlorite filler that has been heat modified by calcining at 950° C.

FIG. 4a is a thermal gravimetric-differential thermal analysis trace for virgin talc powder.

FIG. 4b is a thermal gravimetric-differential thermal analysis trace for a talc filler that has been heat modified by flame fusing.

### DETAILED DESCRIPTION OF THE PREFERRED EMBODIMENTS

The orthodontic compositions described herein are composite materials, each comprising a heat-modified filler, a hardenable component, and a hardener. Optionally, the orthodontic composition includes an additive that provides a secondary function such fluoride release or a color change feature. Hardenable and hardened compositions as described herein can be used for a variety of dental and orthodontic applications that use a material capable of adhering (e.g., bonding) to a tooth structure. While particularly useful as orthodontic adhesives, these hardenable and hardened compositions may also be used, for example, as dental composites, dental adhesives, cements (e.g., glass ionomer cements, resin-modified glass ionomer cements, and orthodontic cements), and combinations thereof Each of the above components (fillers, hardenable components, hardeners, and optional additives) shall be highlighted and described in greater detail in the sub-headings that follow.

### Fillers

### Heat-Modified Fillers

The orthodontic compositions disclosed in the present invention include inorganic fillers that are heat-modified. As used herein, the term “heat-modified” denotes that the filler material has been subjected to a sufficient amount heat to irreversibly eliminate some or all water of hydration from the material to form a non-hydrated phase. As used herein, heat modification specifically excludes drying steps, in which heat causes uncomplexed moisture, or “pore water”, to be evolved from the structure of a material.

Heat-modification can occur, for example, by a flame-fusing process in which particles are dispersed in a combustible gas mixture and passed through a flame front. This process allows partial fusion to occur within at least the surfaces of the particles at high thermal efficiency, while particle agglomeration during fusion is inhibited. Flame fusing can be used to convert filler particles that are generally plate-shaped, such as talc or chlorite, into ones that have a generally ellipsoidal shape. This can be advantageously used to tailor the handling properties of a composite that uses these filler particles. Other controlled methods capable of melting or softening small particles to form generally ellipsoidal particles include atomization, fire polishing, and direct fusion. Each of these processes is described in detail in U.S. Pat. No. 6,045,913 (Castle).

An alternative method of heat-modification is calcining As used herein, “calcining” is a thermal treatment process applied to a solid material in order to bring about a thermal decomposition or a phase transition reaction. Calcining is typically conducted at a high temperature but below the melting or fusing point of the material. When using calcining to heat modify an inorganic mineral filler, that the mineral is preferably heated above its dissociation temperature for eliminating water of hydration. This temperature varies somewhat from one mineral to another, and examples of dissociation temperatures for representative materials are provided in Table I on p. 86 of Parmelee, C., Ceramic Glazes, Industrial Publications, 2^{nd} Edition, 1951. Based on scanning electron microscopy of calcined filler materials, calcining is different from flame fusion in that it does not appear to significantly affect overall distribution of filler particle size or shape.

Heat modification of the mineral fillers can provide certain benefits. Moisture contamination is known to reduce bond strength in orthodontic bonding. Since heat modification reduces the amount of complexed moisture present in the mineral filler particles, it is possible that the reduction of complexed moisture may enhance bond strength. The presence of heat may also densify the mineral particles, rendering them harder, tougher, and more resistant to cohesive failure. Densification of filler particles can therefore provide greater reinforcement to the resin matrix material.

Further, in the case where heat modification is achieved through flame fusing, at least some of the mineral particles are transformed from plate-shaped particles to elliptical particles. This has been empirically found to provide the additional unexpected benefit of increasing maximum filler loading, or the extent to which the filler can be loaded into a composite paste before the paste becomes unstable and phase separation occurs (see later Examples 1-2 and Comparative Examples CE-1, CE-2). Increasing the maximum filler loading is advantageous because it provides greater flexibility in both the design and manufacture of an orthodontic composition. Filler loading also positively correlates with filler packing density, which could influence the bond strength and cohesive strength of the hardened orthodontic composition. The relationship between filler packing density and overall strength has been well documented for composites in general.

Various inorganic filler materials may be heat modified. Examples of inorganic filler materials include, but are not limited to: quartz (i.e., silica, SiO₂); nitrides (e.g., silicon nitride); glasses and fillers derived from, for example, Zr, Sr, Ce, Sb, Sn, Ba, Zn, and Al; feldspar; borosilicate glass; kaolin; talc; zirconia; titania; low Mohs hardness fillers such as those described in U.S. Pat. No. 4,435,160 (Randklev), U.S. Pat. No. 4,695,251 (Randklev), and U.S. Pat. No. 4,906,185 (Randklev); and submicron silica particles (e.g., pyrogenic silicas such as those provided under the trade designations AEROSIL, including “OX 50,” “130,” “150” and “200” silicas from Degussa Corp., Akron, Ohio and CAB-O-SIL M5 and TS-720 silica from Cabot Corp., Tuscola, Ill.). Preferably, the inorganic filler has a color that generally matches that of human enamel and is free of any contaminants, such as graphite, that may discolor the filler and produce a non-aesthetic result. It is also preferred that the filler selected does not significantly stain or change color as a result of absorption or reaction with adhesive components, orthodontic appliance materials, oral fluids or food substances. This is particularly relevant when the composition is used for bonding aesthetic, translucent appliances such as CLARITY brand ceramic brackets from 3M Unitek (Monrovia, Calif.).

Preferred heat-modified mineral fillers have a Mohs hardness not exceeding 5 to facilitate removal of remnant hardened composition. These fillers include, for example, talc, barite, aragonite, calcite and chlorite. It is noted that human enamel has a Mohs hardness ranging from 4.5 to 5, depending on the age and location of the associated tooth. To provide the greatest flexibility in selecting a suitable abrasive system to remove the remnant, it is preferred to select a filler that is significantly softer than the enamel—for example having a Mohs hardness of 4 or lower.

In some embodiments, the heat-modified mineral filler has a controlled particle size (the largest dimension of a particle, typically the diameter) and/or particle size distribution. Particles may be flat, ellipsoidal, spherical, rod-like, or some other asymmetrical shape. The particle size distribution may be unimodal or polymodal (e.g., bimodal). Preferably, the median particle size of the filler is 25 micrometers or less, more preferably 20 micrometers or less, and most preferably 15 micrometers or less. Preferably, the median particle size of the filler is greater than 1 micrometer, more preferably greater than 2 micrometers, and most preferably greater than 4 micrometers.

The fillers used in this invention can also be characterized by their “loss on ignition”. Loss on ignition is a test that can help identify the presence of hydrates or labile hydroxy-compounds. The test consists of strongly heating (or igniting) a sample of the material at a specified temperature, and allowing volatile substances to escape, until its mass ceases to change. This may be done in air, or in some other reactive or inert atmosphere. The simple test typically consists of placing a few grams of the material in a weighed, pre-ignited crucible and determining its mass, placing it in a temperature-controlled furnace for a set time, cooling it in a controlled (e.g. water-free, CO₂-free) atmosphere, and redetermining the mass. The process may be repeated to show that mass-change is complete. In some embodiments, the inorganic mineral filler has a loss on ignition prior to heat-modification ranging from 10 to 15 percent by weight.

A variant of the test in which weight change is continually monitored as temperature is changed is thermal gravimetric analysis, or TGA. This technique is capable of detecting the presence of phase transitions, gas evolution, oxidation processes, hydration, and various other reactions that take place as a material is heated or cooled. During the test, the weight gain or loss is continually recorded and converted to a weight percent change on the Y-axis against the sample material temperature on the X-axis. TGA instruments are sometimes also equipped with differential thermal analysis (DTA) capability. In DTA, heat flow is simultaneously monitored and recorded as the sample material is heated or cooled. This way, it is possible to measure not only the changes in weight that occur, but also the release or consumption of heat when exothermic or endothermic processes occur, respectively. TGA and DTA can be useful in quantifying the amount of hydrated phase present and distinguishing between heat-modified and virgin filler materials, as will be described further in the EXAMPLES section.

The heat-modified filler is preferably present in the orthodontic composition in a range from 50 percent by weight to 75 percent by weight, and more preferably from 66 percent by weight to 72 percent by weight, based on the overall weight of the composition. Preferably less than 5 weight percent of the filler is hydrated and more preferably less than 3 weight percent of the filler is hydrated, based on the overall weight of the filler. Most preferably, essentially none of the filler is hydrated; for example, TGA shows no hydrated phase is present in the heat-modified filler. Blends of two or more heat-modified inorganic fillers can also be used.

### Hybrid Fillers

Heat-modified inorganic fillers may be blended with other fillers to form a hybrid filler system. For example, the heat-modified inorganic filler may be mixed with virgin inorganic fillers or even organic fillers. The inclusion of an inorganic filler component may be used to impart desirable set of properties or features to the composition not possible using a homogenous filler system. As will be described in more detail later, for example, inorganic fillers can be used to impart a fluoride releasing property to the composition. Fluoride release is a desirable feature to many orthodontists. Inorganic fillers may also be effective in increasing the modulus or strength of the composition or modifying its rheological properties to optimize handling by the clinician or orthodontic assistant.

Organic fillers such as polymeric fillers are generally soft and can be advantageously included in a filler mixture without significantly compromising the ease of cleanup. Exemplary polymeric fillers include those composed of natural and synthetic polymers and copolymers, such as polymethacrylic polymers, polystyrene, polycyanoacrylates, polytetrafluoroethylene, polycarbonates, polyamides, nylons, polyester, polyolefin, polyvinylchloride, polyepoxides and polyurethanes. Preferred polymeric fillers are disclosed in co-owned and copending U.S. Provisional Patent Application Ser. No. 60/976501 (Kalgutkar et al), filed on Sep. 30, 2007.

The amounts in which additional fillers may be added to the composition, however, depend in part on the nature of the inorganic filler. If relatively soft inorganic fillers are selected, significant amounts may be tolerated in the composition without unduly compromising ease of remnant cleanup. Hard fillers, however, such as those with a Mohs hardness of at least 5, may adversely impact the ease of remnant cleanup if added in significant quantities. In cases where inorganic fillers have a Mohs hardness of at least 5, it is preferred that the inorganic component constitutes 5 percent or less of the total weight of the composition. More preferably, any inorganic components with a Mohs hardness of at least 5 constitutes 2 percent or less of the total weight of the composition. Most preferably, and for the reasons set out above, the composition does not contain any fillers with a Mohs hardness of at least 5. In certain embodiments, the composition does not contain any fillers with a Mohs hardness of at least 6, or fillers with a Mohs hardness of at least 7, or fillers with a Mohs hardness of at least 8, or fillers with a Mohs hardness of at least 9. The filler should in any event be nontoxic and suitable for use in the mouth. The filler can be radiopaque or radiolucent. The filler typically is substantially insoluble in water.

Preferred non-acid-reactive filler particles include quartz (i.e., silica), submicron silica, zirconia, submicron zirconia, and non-vitreous microparticles of the type described in U.S. Pat. No. 4,503,169 (Randklev). Mixtures of these non-acid-reactive fillers are also contemplated, as well as combination fillers made from organic and inorganic materials.

The filler can also be an acid-reactive filler. Suitable acid-reactive fillers include metal oxides, glasses, and metal salts. Typical metal oxides include barium oxide, calcium oxide, magnesium oxide, and zinc oxide. Typical glasses include borate glasses, phosphate glasses, and fluoroaluminosilicate (“FAS”) glasses. FAS glasses are particularly preferred. The FAS glass typically contains sufficient elutable cations so that a hardened composition will form when the glass is mixed with the components of the composition. In fluoride releasing compositions, the glass typically contains sufficient elutable fluoride ions so that the hardened composition will have cariostatic properties. The glass can be made from a melt containing fluoride, alumina, and other glass-forming ingredients using techniques familiar to those skilled in the FAS glassmaking art. The FAS glass typically is in the form of particles that are sufficiently finely divided so that they can conveniently be mixed with other components and will perform well when the resulting mixture is used in the mouth. Preferably, the FAS glass is present in the composition in an amount up to 40% by weight, based on the overall weight of the composition. The total filler content (having a Mohs hardness below 5) is preferably present in a range from 50 percent by weight to 75 percent by weight, and more preferably from 66 percent by weight to 72 percent by weight, based on the overall weight of the composition.

Generally, the average particle size for the FAS glass is no greater than 12 micrometers, typically no greater than 10 micrometers, and more typically no greater than 5 micrometers as measured using, for example, a sedimentation analyzer. Suitable FAS glasses will be familiar to those skilled in the art, and are provided from a wide variety of commercial sources, and many are found in glass ionomer cements such as those commercially sold under the trade designations VITREMER, VITREBOND, RELY X LUTING CEMENT, RELY X LUTING PLUS CEMENT, PHOTAC-FIL QUICK, KETAC-MOLAR, and KETAC-FIL PLUS (from 3M ESPE Dental Products, St. Paul, Minn.), FUJI II LC and FUJI IX (G-C Dental Industrial Corp., Tokyo, Japan) and CHEMFIL Superior (Dentsply International, York, Pa.). Mixtures of fillers can be used if desired. Further description of other compositions containing FAS glass filler is provided under the HARDENABLE COMPONENTS/GLASS IONOMERS sub-heading below.

The surface of the filler particles can also be treated with a coupling agent in order to enhance the bond between the filler and the resin. Suitable coupling agents include gamma-methacryloxypropyltrimethoxysilane, gamma-mercaptopropyltriethoxysilane, gamma-aminopropyltrimethoxysilane, and the like. Silane-treated talc filler, silane-treated kaolin filler, silane-treated clay-based filler, and combinations thereof are especially preferred in certain embodiments.

Other suitable fillers are disclosed in U.S. Pat. No. 6,387,981 (Zhang et al.) and U.S. Pat. No. 6,572,693 (Wu et al.) as well as International Patent Application Publication Nos. WO 01/30305 (Zhang et al.), WO 01/30306 (Windisch et al.), WO 01/30307 (Zhang et al.), and WO 03/063804 (Wu et al.). Filler components described in these references include nanosized silica particles, nanosized metal oxide particles, and combinations thereof Nanofillers are also described in U.S. Pat. No. 7,090,721 (Craig et al.), U.S. Pat. No. 7,156,911 (Kangas et al.) and U.S. Pat. No. 7,090,722 (Budd et al.); and U.S. Patent Application Publication No. 2005/0256223 A1 (Kolb et al.).

### Hardenable Components

### Ethylenically Unsaturated Compounds

As disclosed herein, suitable compositions may use hardenable components (e.g., photopolymerizable compounds) including ethylenically unsaturated compounds (which contain free radically active unsaturated groups). In certain embodiments of the present invention, hardenable components are preferably present in a range from 20% to 60% by weight and more preferably in a range from 30% to 45% by weight, based on the overall weight of the composition. Examples of useful ethylenically unsaturated compounds include acrylic acid esters, methacrylic acid esters, hydroxy-functional acrylic acid esters, hydroxy-functional methacrylic acid esters, and combinations thereof.

The compositions (e.g., photopolymerizable compositions) may include compounds having free radically active functional groups that may include monomers, oligomers, and polymers having one or more ethylenically unsaturated group. Suitable compounds contain at least one ethylenically unsaturated bond and are capable of undergoing addition polymerization. Such free radically polymerizable compounds include mono-, di- or poly-(meth)acrylates (i.e., acrylates and methacrylates) such as, methyl(meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl(meth)acrylate, bis[1-(2-acryloxy)]-p-ethoxyphenyldimethylmethane, bis[1-(3-acryloxy-2-hydroxy)]p-propoxyphenyldimethylmethane, ethoxylated bisphenol A di(meth)acrylate, and trishydroxyethyl-isocyanurate trimethacrylate; (meth)acrylamides (i.e., acrylamides and methacrylamides) such as (meth)acrylamide, methylene bis-(meth)acrylamide, and diacetone(meth)acrylamide; urethane(meth)acrylates; the bis-(meth)acrylates of polyethylene glycols (preferably of molecular weight 200-500), copolymerizable mixtures of acrylated monomers such as those in U.S. Pat. No. 4,652, 274 (Boettcher et al.), acrylated oligomers such as those of U.S. Pat. No. 4,642,126 (Zador et al.), and poly(ethylenically unsaturated) carbamoyl isocyanurates such as those disclosed in U.S. Pat. No. 4,648,843 (Mitra); and vinyl compounds such as styrene, diallyl phthalate, divinyl succinate, divinyl adipate and divinyl phthalate. Other suitable free radically polymerizable compounds include siloxane-functional (meth)acrylates as disclosed, for example, in WO-00/38619 (Guggenberger et al.), WO-01/92271 (Weinmann et al.), WO-01/07444 (Guggenberger et al.), WO-00/42092 (Guggenberger et al.) and fluoropolymer-functional (meth)acrylates as disclosed, for example, in U.S. Pat. No. 5,076,844 (Fock et al.), U.S. Pat. No. 4,356,296 (Griffith et al.), EP-0373 384 (Wagenknecht et al.), EP-0201 031 (Reiners et al.), and EP-0201 778 (Reiners et al.). Mixtures of two or more free radically polymerizable compounds can be used if desired.

The hardenable component may also contain hydroxyl groups and ethylenically unsaturated groups in a single molecule. Examples of such materials include hydroxyalkyl(meth)acrylates, such as 2-hydroxyethyl (meth)acrylate and 2-hydroxypropyl(meth)acrylate; glycerol mono- or di-(meth)acrylate; trimethylolpropane mono- or di-(meth)acrylate; pentaerythritol mono-, di-, and tri-(meth)acrylate; sorbitol mono-, di-, tri-, tetra-, or penta-(meth)acrylate; and 2,2-bis[4-(2-hydroxy-3-ethacryloxypropoxy)phenyl]propane (bisGMA). Suitable ethylenically unsaturated compounds are also provided from a wide variety of commercial sources, such as Sigma-Aldrich, St. Louis. Mixtures of ethylenically unsaturated compounds can be used if desired.

In certain embodiments hardenable components include PEGDMA (polyethyleneglycol dimethacrylate having a molecular weight of approximately 400), bisGMA, UDMA (urethane dimethacrylate), GDMA (glycerol dimethacrylate), TEGDMA (triethyleneglycol dimethacrylate), bisEMA6 as described in U.S. Pat. No. 6,030,606 (Holmes), and NPGDMA (neopentylglycol dimethacrylate). Various combinations of the hardenable components can be used if desired. In certain embodiments, crosslinking monomers such as UDMA may represent the entire hardenable component of the composition. Preferably compositions as disclosed herein include at least 10% by weight, preferably at least 20% by weight, and more preferably at least 30% by weight ethylenically unsaturated compounds (e.g., with and/or without acid functionality), based on the overall weight of the composition. Moreover, compositions as disclosed herein include at most 60% by weight, preferably at most 50% by weight, and more preferably at most 45% by weight ethylenically unsaturated compounds (e.g., with and/or without acid functionality), based on the overall weight of the composition.

Compositions as disclosed herein may also include one or more hardenable components in the form of ethylenically unsaturated compounds with acid functionality. As used herein, ethylenically unsaturated compounds with acid functionality is meant to include monomers, oligomers, and polymers having ethylenic unsaturation and acid and/or acid-precursor functionality. Acid-precursor functionalities include, for example, anhydrides, acid halides, and pyrophosphates. The acid functionality can include carboxylic acid functionality, phosphoric acid functionality, phosphonic acid functionality, sulfonic acid functionality, or combinations thereof.

Ethylenically unsaturated compounds with acid functionality include, for example, α,β-unsaturated acidic compounds such as glycerol phosphate mono(meth)acrylates, glycerol phosphate di(meth)acrylates, hydroxyethyl(meth)acrylate (e.g., HEMA) phosphates, bis((meth)acryloxyethyl)phosphate, ((meth)acryloxypropyl)phosphate, bis((meth)acryloxypropyl)phosphate, bis((meth)acryloxy)propyloxy phosphate, (meth)acryloxyhexyl phosphate, bis((meth)acryloxyhexyl)phosphate, (meth)acryloxyoctyl phosphate, bis((meth)acryloxyoctyl) phosphate, (meth)acryloxydecyl phosphate, bis((meth)acryloxydecyl)phosphate, caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like, may be used as components in the hardenable component system. Also monomers, oligomers, and polymers of unsaturated carbonic acids such as (meth)acrylic acids, aromatic (meth)acrylated acids (e.g., methacrylated trimellitic acids), and anhydrides thereof can be used. Certain compositions for use in preferred embodiments of the present invention include an ethylenically unsaturated compound with acid functionality having at least one P—OH moiety.

Certain of these compounds are obtained, for example, as reaction products between isocyanatoalkyl(meth)acrylates and carboxylic acids. Additional compounds of this type having both acid-functional and ethylenically unsaturated components are described in U.S. Pat. No. 4,872,936 (Engelbrecht) and U.S. Pat. No. 5,130,347 (Mitra). A wide variety of such compounds containing both the ethylenically unsaturated and acid moieties can be used. Mixtures of such compounds can be used if desired.

Additional ethylenically unsaturated compounds with acid functionality include, for example, polymerizable bisphosphonic acids as disclosed for example, in U.S. Patent Application Publication No. 2004/0206932 (Abuelyaman et al.); AA:ITA:IEM (copolymer of acrylic acid:itaconic acid with pendent methacrylate made by reacting AA:ITA copolymer with sufficient 2-isocyanatoethyl methacrylate to convert a portion of the acid groups of the copolymer to pendent methacrylate groups as described, for example, in Example 11 of U.S. Pat. No. 5,130,347 (Mitra)); and those recited in U.S. Pat. No. 4,259,075 (Yamauchi et al.), U.S. Pat. No. 4,499,251 (Omura et al.), U.S. Pat. No. 4,537,940 (Omura et al.), U.S. Pat. No. 4,539,382 (Omura et al.), U.S. Pat. No. 5,530,038 (Yamamoto et al.), U.S. Pat. No. 6,458,868 (Okada et al.), and European Patent Application Publication Nos. EP 712,622 (Tokuyama Corp.) and EP 1,051,961 (Kuraray Co., Ltd.).

Compositions as disclosed herein can also include compositions that include combinations of ethylenically unsaturated compounds with acid functionality. Preferably the compositions are self-adhesive and are non-aqueous. For example, such compositions can include: a first compound including at least one (meth)acryloxy group and at least one —O—P(O)(OH)ₓ group, wherein x=1 or 2, and wherein the at least one —O—P(O)(OH)ₓ group and the at least one (meth)acryloxy group are linked together by a C1-C4 hydrocarbon group; a second compound including at least one (meth)acryloxy group and at least one —O—P(O)(OH)ₓ group, wherein x=1 or 2, and wherein the at least one —O—P(O)(OH)ₓ group and the at least one (meth)acryloxy group are linked together by a C5-C12 hydrocarbon group; an ethylenically unsaturated compound without acid functionality; an initiator system; and a filler. Such compositions are described, for example, in U.S. Provisional Application Ser. No. 60/600,658 (Luchterhandt et al.), filed on Aug. 11, 2004. Preferably compositions as disclosed herein include at least 10% by weight, preferably at least 20% by weight, and more preferably at least 30% by weight ethylenically unsaturated compounds with acid functionality, based on the overall weight of the composition. Compositions as disclosed herein include at most 60% by weight, preferably at most 50% by weight, and more preferably at most 45% by weight ethylenically unsaturated compounds with acid functionality, based on the total weight of the composition.

### Epoxy (Oxirane) or Vinyl Ether Compounds

Compositions as disclosed herein may also include one or more hardenable components in the form of epoxy (oxirane) compounds (which contain cationically active epoxy groups) or vinyl ether compounds (which contain cationically active vinyl ether groups).

The epoxy or vinyl ether monomers can be used alone as the hardenable component in a composition or in combination with other monomer classes, e.g., ethylenically unsaturated compounds as described herein, and can include as part of their chemical structures aromatic groups, aliphatic groups, cycloaliphatic groups, and combinations thereof.

Examples of epoxy (oxirane) compounds include organic compounds having an oxirane ring that is polymerizable by ring opening. These materials include monomeric epoxy compounds and epoxides of the polymeric type and can be aliphatic, cycloaliphatic, aromatic or heterocyclic. These compounds generally have, on the average, at least 1 polymerizable epoxy group per molecule, in some embodiments at least 1.5, and in other embodiments at least 2 polymerizable epoxy groups per molecule. The polymeric epoxides include linear polymers having terminal epoxy groups (e.g., a diglycidyl ether of a polyoxyalkylene glycol), polymers having skeletal oxirane units (e.g., polybutadiene polyepoxide), and polymers having pendent epoxy groups (e.g., a glycidyl methacrylate polymer or copolymer). The epoxides may be pure compounds or may be mixtures of compounds containing one, two, or more epoxy groups per molecule. The “average” number of epoxy groups per molecule is determined by dividing the total number of epoxy groups in the epoxy-containing material by the total number of epoxy-containing molecules present.

These epoxy-containing materials may vary from low molecular weight monomeric materials to high molecular weight polymers and may vary greatly in the nature of their backbone and substituent groups. Illustrative of permissible substituent groups include halogens, ester groups, ethers, sulfonate groups, siloxane groups, carbosilane groups, nitro groups, phosphate groups, and the like. The molecular weight of the epoxy-containing materials may vary from 58 to 100,000 or more.

Suitable epoxy-containing materials useful as the resin system reactive components for use in certain embodiments of the present invention are listed in U.S. Pat. No. 6,187,836 (Oxman et al.) and U.S. Pat. No. 6,084,004 (Weinmann et al.).

Other suitable epoxy resins useful as the resin system reactive components include those which contain cyclohexene oxide groups such as epoxycyclohexanecarboxylates, typified by 3,4-epoxycyclohexylmethyl-3,4-epoxycyclohexanecarboxylate, 3,4-epoxy-2-methylcyclohexylmethyl-3,4-epoxy-2-methylcyclohexane carboxylate, and bis(3,4-epoxy-6-methylcyclohexyl-methyl) adipate. For a more detailed list of useful epoxides of this nature, reference is made to U.S. Pat. No. 6,245,828 (Weinmann et al.) and U.S. Pat. No. 5,037,861 (Crivello et al.); and U.S. Pat. No. 6,779,656 (Klettke et al.).

Other epoxy resins that may be useful in compositions as disclosed herein include glycidyl ether monomers. Examples are glycidyl ethers of polyhydric phenols obtained by reacting a polyhydric phenol with an excess of chlorohydrin such as epichlorohydrin (e.g., the diglycidyl ether of 2,2-bis-(2,3-epoxypropoxyphenol)propane). Further examples of epoxides of this type are described in U.S. Pat. No. 3,018,262 (Schroeder).

Other suitable epoxides useful as the resin system reactive components are those that contain silicon, useful examples of which are described in International Patent Application Publication No. WO 01/51540 (Klettke et al.).

Additional suitable epoxides useful as the resin system reactive components include octadecylene oxide, epichlorohydrin, styrene oxide, vinyl cyclohexene oxide, glycidol, glycidylmethacrylate, diglycidyl ether of Bisphenol A and other commercial epoxides, as provided in U.S. Pat. No. 7,262,228 (Oxman et al.).

Blends of various epoxy-containing materials are also contemplated. Examples of such blends include two or more weight average molecular weight distributions of epoxy-containing compounds, such as low molecular weight (below 200), intermediate molecular weight (200 to 10,000) and higher molecular weight (above 10,000). Alternatively or additionally, the epoxy resin may contain a blend of epoxy-containing materials having different chemical natures, such as aliphatic and aromatic, or functionalities, such as polar and non-polar.

Other types of useful hardenable components having cationically active functional groups include vinyl ethers, oxetanes, spiro-orthocarbonates, spiro-orthoesters, and the like.

If desired, both cationically active and free radically active functional groups may be contained in a single molecule. Such molecules may be obtained, for example, by reacting a di- or poly-epoxide with one or more equivalents of an ethylenically unsaturated carboxylic acid. An example of such a material is the reaction product of UVR-6105 (from Dow Chemical Company in Midland, Mich.) with one equivalent of methacrylic acid. Commercial materials having epoxy and free-radically active functionalities include the CYCLOMER series, such as CYCLOMER M-100, M-101, or A-200 from Daicel Chemical, Japan, and EBECRYL-3605 from Radcure Specialties, UCB Chemicals, Atlanta, Ga.

The cationically curable components may further include a hydroxyl-containing organic material. Suitable hydroxyl-containing materials may be any organic material having hydroxyl functionality of at least 1, and preferably at least 2. Preferably, the hydroxyl-containing material contains two or more primary or secondary aliphatic hydroxyl groups (i.e., the hydroxyl group is bonded directly to a non-aromatic carbon atom). The hydroxyl groups can be terminally situated, or they can be pendent from a polymer or copolymer. The molecular weight of the hydroxyl-containing organic material can vary from very low (e.g., 32) to very high (e.g., one million or more). Suitable hydroxyl-containing materials can have low molecular weights (i.e., from 32 to 200), intermediate molecular weights (i.e., from 200 to 10,000, or high molecular weights (i.e., above 10,000). As used herein, all molecular weights are weight average molecular weights.

The hydroxyl-containing materials may be non-aromatic in nature or may contain aromatic functionality. The hydroxyl-containing material may optionally contain heteroatoms in the backbone of the molecule, such as nitrogen, oxygen, sulfur, and the like. The hydroxyl-containing material may, for example, be selected from naturally occurring or synthetically prepared cellulosic materials. The hydroxyl-containing material should be substantially free of groups which may be thermally or photolytically unstable; that is, the material should not decompose or liberate volatile components at temperatures below 100° C. or in the presence of actinic light which may be encountered during the desired photopolymerization conditions for the polymerizable compositions. Suitable hydroxyl-containing materials useful in certain embodiments of the present invention are listed in U.S. Pat. No. 6,187,836 (Oxman et al.).

The hardenable component(s) may also contain hydroxyl groups and cationically active functional groups in a single molecule. An example is a single molecule that includes both hydroxyl groups and epoxy groups.

### Glass Ionomers

Compositions as described herein may include glass ionomer cements such as conventional glass ionomer cements that typically employ as their main ingredients a homopolymer or copolymer of an ethylenically unsaturated carboxylic acid (e.g., poly acrylic acid, copoly (acrylic, itaconic acid), and the like), a fluoroaluminosilicate (“FAS”) glass, water, and a chelating agent such as tartaric acid. Conventional glass ionomers (i.e., glass ionomer cements) typically are supplied in powder/liquid formulations that are mixed just before use. The mixture will undergo self-hardening in the dark due to an ionic reaction between the acidic repeating units of the polycarboxylic acid and cations leached from the glass.

The glass ionomer cements may also include resin-modified glass ionomer (“RMGI”) cements. Like a conventional glass ionomer, an RMGI cement employs an FAS glass. However, the organic portion of an RMGI is different. In one type of RMGI, the polycarboxylic acid is modified to replace or end-cap some of the acidic repeating units with pendent curable groups and a photoinitiator is added to provide a second cure mechanism, e.g., as described in U.S. Pat. No. 5,130,347 (Mitra). Acrylate or methacrylate groups are usually employed as the pendant curable group. In another type of RMGI, the cement includes a polycarboxylic acid, an acrylate or methacrylate-functional monomer and a photoinitiator, e.g., as in Mathis et al., “Properties of a New Glass Ionomer/Composite Resin Hybrid Restorative”, Abstract No. 51, J. Dent Res., 66:113 (1987) and as in U.S. Pat. No. 5,063,257 (Akahane et al.), U.S. Pat. No. 5,520,725 (Kato et al.), U.S. Pat. No. 5,859,089 (Qian), U.S. Pat. No. 5,925,715 (Mitra) and U.S. Pat. No. 5,962,550 (Akahane et al.). In another type of RMGI, the cement may include a polycarboxylic acid, an acrylate or methacrylate-functional monomer, and a redox or other chemical cure system, e.g., as described in U.S. Pat. No. 5,154,762 (Mitra et al.), U.S. Pat. No. 5,520,725 (Kato et al.), and U.S. Pat. No. 5,871,360 (Kato). In another type of RMGI, the cement may include various monomer-containing or resin-containing components as described in U.S. Pat. No. 4,872,936 (Engelbrecht), U.S. Pat. No. 5,227,413 (Mitra), U.S. Pat. No. 5,367,002 (Huang et al.), and U.S. Pat. No. 5,965,632 (Orlowski). RMGI cements are preferably formulated as powder/liquid or paste/paste systems, and contain water as mixed and applied. The compositions are able to harden in the dark due to the ionic reaction between the acidic repeating units of the polycarboxylic acid and cations leached from the glass, and commercial RMGI products typically also cure on exposure of the cement to light from a dental curing lamp. RMGI cements that contain a redox cure system and that can be cured in the dark without the use of actinic radiation are described in U.S. Pat. No. 6,765,038 (Mitra).

### Hardeners

### Photoinitiator Systems

In certain embodiments, the compositions of the present invention are photopolymerizable, i.e., the hardenable component is photopolymerizable and the hardener includes a photoinitiator (or photoinitiator system), in which irradiation with actinic radiation initiates the polymerization (or hardening) of the composition. Such photopolymerizable compositions can be hardened by free radical polymerization or cationic polymerization.

Suitable photoinitiators (i.e., photoinitiator systems that include one or more compounds) for inducing free radical photopolymerization include binary and tertiary systems. Typical tertiary photoinitiators include an iodonium salt, a photosensitizer, and an electron donor compound as described in U.S. Pat. No. 5,545,676 (Palazzotto et al.). Preferred iodonium salts are the diaryl iodonium salts, e.g., diphenyliodonium chloride, diphenyliodonium hexafluorophosphate, diphenyliodonium tetrafluoroborate, and tolylcumyliodonium tetrakis(pentafluorophenyl)borate. Preferred photosensitizers are monoketones and diketones that absorb some light within a range of 400 nm to 520 nm (preferably, 450 nm to 500 nm). More preferred compounds are alpha diketones that have some light absorption within a range of 400 nm to 520 nm (even more preferably, 450 to 500 nm). Preferred compounds are camphorquinone, benzil, furil, 3,3,6,6-tetramethylcyclohexanedione, phenanthraquinone, 1-phenyl-1,2-propanedione and other 1-aryl-2-alkyl-1,2-ethanediones, and cyclic alpha diketones. Most preferred is camphorquinone. Preferred electron donor compounds include substituted amines, e.g., ethyl dimethylaminobenzoate. Other suitable tertiary photoinitiator systems useful for photopolymerizing cationically polymerizable resins are described, for example, in U.S. Pat. No. 6,765,036 (Dede et al.).

Other suitable photoinitiators for polymerizing free radically photopolymerizable compositions include the class of phosphine oxides that typically have a functional wavelength range of 380 nm to 1200 nm. Preferred phosphine oxide free radical initiators with a functional wavelength range of 380 nm to 450 nm are acyl and bisacyl phosphine oxides such as those described in U.S. Pat. No. 4,298,738 (Lechtken et al.), U.S. Pat. No. 4,324,744 (Lechtken et al.), U.S. Pat. No. 4,385,109 (Lechtken et al.), U.S. Pat. No. 4,710,523 (Lechtken et al.), and U.S. Pat. No. 4,737,593 (Ellrich et al.), U.S. Pat. No. 6,251,963 (Kohler et al.); and EP Application No. 0 173 567 A2 (Ying).

Commercial phosphine oxide photoinitiators capable of free-radical initiation when irradiated at wavelength ranges of greater than 380 nm to 450 nm include bis(2,4,6-trimethylbenzoyl)phenyl phosphine oxide (IRGACURE 819, Ciba Specialty Chemicals, Tarrytown, N.Y.), bis(2,6-dimethoxybenzoyl)-(2,4,4-trimethylpentyl) phosphine oxide (CGI 403, Ciba Specialty Chemicals), a 25:75 mixture, by weight, of bis(2,6-dimethoxybenzoyl)-2,4,4-trimethylpentyl phosphine oxide and 2-hydroxy-2-methyl-1-phenylpropan-1-one (IRGACURE 1700, Ciba Specialty Chemicals), a 1:1 mixture, by weight, of bis(2,4,6-trimethylbenzoyl)phenyl phosphine oxide and 2-hydroxy-2-methyl-1-phenylpropane-1-one (DAROCUR 4265, Ciba Specialty Chemicals), and ethyl 2,4,6-trimethylbenzylphenyl phosphinate (LUCIRIN LR8893X, BASF Corp., Charlotte, N.C.).

Typically, the phosphine oxide initiator is present in the photopolymerizable composition in catalytically effective amounts, such as from 0.1 weight percent to 5.0 weight percent, based on the total weight of the composition.

Tertiary amine reducing agents may be used in combination with an acylphosphine oxide. Illustrative tertiary amines useful in certain embodiments of the invention include ethyl 4-(N,N-dimethylamino)benzoate and N,N-dimethylaminoethyl methacrylate. When present, the amine reducing agent is present in the photopolymerizable composition in an amount from 0.1 weight percent to 5.0 weight percent, based on the total weight of the composition. Useful amounts of other initiators are well known to those of skill in the art.

Suitable photoinitiators for polymerizing cationically photopolymerizable compositions include binary and tertiary systems. Typical tertiary photoinitiators include an iodonium salt, a photosensitizer, and an electron donor compound as described in EP 0 897 710 (Weinmann et al.); in U.S. Pat. No. 5,856,373 (Kaisaki et al.), U.S. Pat. No. 6,084,004 (Weinmann et al.), U.S. Pat. No. 6,187,833 (Oxman et al.), and U.S. Pat. No. 6,187,836 (Oxman et al.); and in U.S. Pat. No. 6,765,036 (Dede et al.). The compositions of certain embodiments of the invention can include one or more anthracene-based compounds as electron donors. In some embodiments, the compositions comprise multiple substituted anthracene compounds or a combination of a substituted anthracene compound with unsubstituted anthracene. The combination of these mixed-anthracene electron donors as part of a photoinitiator system provides significantly enhanced cure depth and cure speed and temperature insensitivity when compared to comparable single-donor photoinitiator systems in the same matrix. Such compositions with anthracene-based electron donors are described in U.S. Pat. No. 7,262,228 (Oxman et al.).

Suitable iodonium salts include tolylcumyliodonium tetrakis(pentafluorophenyl)borate, tolylcumyliodonium tetrakis(3,5-bis(trifluoromethyl)-phenyl)borate, and the diaryl iodonium salts, e.g., diphenyliodonium chloride, diphenyliodonium hexafluorophosphate, diphenyliodonium hexafluoroantimonate, and diphenyliodonium tetrafluoroboarate. Suitable photosensitizers are monoketones and diketones that absorb some light within a range of 450 nm to 520 nm (preferably, 450 nm to 500 nm). More suitable compounds are alpha diketones that have some light absorption within a range of 450 nm to 520 nm (even more preferably, 450 nm to 500 nm). Preferred compounds are camphorquinone, benzil, furil, 3,3,6,6-tetramethylcyclohexanedione, phenanthraquinone and other cyclic alpha diketones. Most preferred is camphorquinone. Suitable electron donor compounds include substituted amines, e.g., ethyl 4-(dimethylamino)benzoate and 2-butoxyethyl 4-(dimethylamino)benzoate; and polycondensed aromatic compounds (e.g. anthracene).

The initiator system, or hardener, is present in an amount sufficient to provide the desired rate of hardening (e.g., polymerizing and/or crosslinking) For a photoinitiator, this amount will be dependent in part on the light source, the thickness of the layer to be exposed to radiant energy, and the extinction coefficient of the photoinitiator. Preferably, the hardener is present in a total amount of at least 0.01% by weight, more preferably, at least 0.03% by weight, and most preferably, at least 0.05% by weight, based on the overall weight of the composition. Preferably, the hardener is present in a total amount of no more than 10% by weight, more preferably, no more than 5% by weight, and most preferably, no more than 2.5% by weight, based on the overall weight of the composition.

### Redox Iniiator Systems

In certain embodiments, the compositions of the present invention are chemically hardenable, i.e., the compositions contain a chemically hardenable component and a chemical initiator (i.e., initiator system) that can polymerize, cure, or otherwise harden the composition without dependence on irradiation with actinic radiation. Such chemical compositions are sometimes referred to as “two-part” or “self-cure” compositions and may include glass ionomer cements, resin-modified glass ionomer cements, redox cure systems, and combinations thereof.

The chemically hardenable compositions may include redox cure systems that include a hardenable component (e.g., an ethylenically unsaturated polymerizable component) and redox agents that include an oxidizing agent and a reducing agent. Examples of hardenable components, redox agents, and optional acid-functional components are described in U.S. Pat. No. 6,982,288 (Mitra et al.) and U.S. Pat. No. 7,173,074 (Mitra et al.).

The reducing and oxidizing agents should react with or otherwise cooperate with one another to produce free-radicals capable of initiating polymerization of the resin system (e.g., the ethylenically unsaturated component or hardenable component). This type of cure is a dark reaction, that is, it is not dependent on the presence of light and can proceed in the absence of light. The reducing and oxidizing agents are preferably sufficiently shelf-stable and free of undesirable colorization to permit their storage and use under typical dental conditions. They should be sufficiently miscible with the resin system (and preferably water-soluble) to permit ready dissolution in (and discourage separation from) the other components of the composition.

Useful reducing agents include ascorbic acid, ascorbic acid derivatives, and metal complexed ascorbic acid compounds as described in U.S. Pat. No. 5,501,727 (Wang et al.); amines, especially tertiary amines, such as 4-tert-butyl dimethylaniline; aromatic sulfinic salts, such as p-toluenesulfinic salts and benzenesulfinic salts; thioureas, such as 1-ethyl-2-thiourea, tetraethyl thiourea, tetramethyl thiourea, 1,1-dibutyl thiourea, and 1,3-dibutyl thiourea; and mixtures thereof. Other secondary reducing agents may include cobalt (II) chloride, ferrous chloride, ferrous sulfate, hydrazine, hydroxylamine (depending on the choice of oxidizing agent), salts of a dithionite or sulfite anion, and mixtures thereof. Preferably, the reducing agent is an amine.

Suitable oxidizing agents will also be familiar to those skilled in the art, and include but are not limited to persulfuric acid and salts thereof, such as sodium, potassium, ammonium, cesium, and alkyl ammonium salts. Additional oxidizing agents include peroxides such as benzoyl peroxides, hydroperoxides such as cumyl hydroperoxide, t-butyl hydroperoxide, and amyl hydroperoxide, as well as salts of transition metals such as cobalt (III) chloride and ferric chloride, cerium (IV) sulfate, perboric acid and salts thereof, permanganic acid and salts thereof, perphosphoric acid and salts thereof, and mixtures thereof.

It may be desirable to use more than one oxidizing agent or more than one reducing agent. Small quantities of transition metal compounds may also be added to accelerate the rate of redox cure. In some embodiments it may be preferred to include a secondary ionic salt to enhance the stability of the polymerizable composition as described in U.S. Pat. No. 6,982,288 (Mitra et al.).

The reducing and oxidizing agents are present in amounts sufficient to permit an adequate free-radical reaction rate. This can be evaluated by combining all of the ingredients of the composition except for the filler, and observing whether or not a hardened mass is obtained.

Preferably, the reducing agent is present in an amount of at least 0.01% by weight, and more preferably at least 0.1% by weight, based on the total weight (including water) of the components of the composition. Preferably, the reducing agent is present in an amount of no greater than 10% by weight, and more preferably no greater than 5% by weight, based on the overall weight (including water) of the composition.

Preferably, the oxidizing agent is present in an amount of at least 0.01% by weight, and more preferably at least 0.10% by weight, based on the total weight (including water) of the components of the composition. Preferably, the oxidizing agent is present in an amount of no greater than 10% by weight, and more preferably no greater than 5% by weight, based on the overall weight (including water) of the composition.

The reducing or oxidizing agents can be microencapsulated as described in U.S. Pat. No. 5,154,762 (Mitra et al.). This will generally enhance shelf stability of the composition, and if necessary permit packaging the reducing and oxidizing agents together. For example, through appropriate selection of an encapsulant, the oxidizing and reducing agents can be combined with an acid-functional component and optional filler and kept in a storage-stable state. Likewise, through appropriate selection of a water-insoluble encapsulant, the reducing and oxidizing agents can be combined with an FAS glass and water and maintained in a storage-stable state.

A redox cure system can be combined with other cure systems, e.g., with a composition such as described U.S. Pat. No. 5,154,762 (Mitra et al.).

### Optional Additives

### Photobleachable and Thermochromic Dyes

In some embodiments, compositions of the present invention preferably have an initial color remarkably different than the color of the patient's tooth. Color is preferably imparted to the composition through the use of a photobleachable or thermochromic dye. The composition preferably includes at least 0.001% by weight photobleachable or thermochromic dye, and more preferably at least 0.002% by weight photobleachable or thermochromic dye, based on the total weight of the composition. The composition preferably includes at most 1% by weight photobleachable or thermochromic dye, and more preferably at most 0.1% by weight photobleachable or thermochromic dye, based on the total weight of the composition. The amount of photobleachable and/or thermochromic dye may vary depending on its extinction coefficient, the ability of the human eye to discern the initial color, and the desired color change. Suitable thermochromic dyes are disclosed, for example, in U.S. Pat. No. 6,670,436 (Burgath et al.).

For embodiments including a photobleachable dye, the color formation and bleaching characteristics of the photobleachable dye varies depending on a variety of factors including, for example, acid strength, dielectric constant, polarity, amount of oxygen, and moisture content in the atmosphere. However, the bleaching properties of the dye can be readily determined by irradiating the composition and evaluating the change in color. Preferably, at least one photobleachable dye is at least partially soluble in a hardenable resin.

Exemplary classes of photobleachable dyes are disclosed, for example, in U.S. Pat. No. 6,331,080 (Cole et al.), U.S. Pat. No. 6,444,725 (Trom et al.), and U.S. Pat. No. 6,528,555 (Nikutowski et al.). Preferred dyes include, for example, Rose Bengal, Methylene Violet, Methylene Blue, Fluorescein, Eosin Yellow, Eosin Y, Ethyl Eosin, Eosin bluish, Eosin B, Erythrosin B, Erythrosin Yellowish Blend, Toluidine Blue, 4′,5′-Dibromofluorescein, and combinations thereof.

The color change in the inventive compositions is initiated by light. Preferably, the composition's color change is initiated using actinic radiation using, for example, a dental curing light which emits visible or near infrared (IR) light for a sufficient amount of time. The mechanism that initiates the color change in the compositions of certain embodiments of the invention may be separate from or substantially simultaneous with the hardening mechanism that hardens the resin. For example, a composition may harden when polymerization is initiated chemically (e.g., redox initiation) or thermally, and the color change from an initial color to a final color may occur subsequent to the hardening process upon exposure to actinic radiation.

The change in composition color from an initial color to a final color is preferably quantified by a color test. Using a color test, a value of ΔE* is determined, which indicates the total color change in a 3-dimensional color space. The human eye can detect a color change of approximately 3 ΔE* units in normal lighting conditions. The compositions of certain embodiments of the present invention are preferably capable of having a color change, ΔE*, of at least 20; more preferably, ΔE* is at least 30; most preferably ΔE* is at least 40.

### Miscellaneous Additives

Optionally, compositions of the present invention may contain solvents (e.g., alcohols (e.g., propanol, ethanol), ketones (e.g., acetone, methyl ethyl ketone), esters (e.g., ethyl acetate), other nonaqueous solvents (e.g., dimethylformamide, dimethylacetamide, dimethylsulfoxide, 1-methyl-2-pyrrolidinone)), and water.

If desired, the compositions of the invention can optionally contain additives such as indicators, dyes, pigments, inhibitors, accelerators, viscosity modifiers, wetting agents, buffering agents, stabilizers, and other similar ingredients that will be apparent to those skilled in the art. Viscosity modifiers include the thermally responsive viscosity modifiers (such as PLURONIC F-127 and F-108 from BASF Wyandotte Corporation, Parsippany, N.J.) and may optionally include a polymerizable moiety on the modifier or a polymerizable component different than the modifier. Such thermally responsive viscosity modifiers are described in U.S. Pat. No. 6,669,927 (Trom et al.) and U.S. Patent Application Publication No. 2004/0151691 (Oxman et al.).

Additionally, medicaments or other therapeutic substances can be optionally added to the compositions. Examples include, but are not limited to, fluoride sources such as tetrabutyl ammonium tetrafluoroborate, whitening agents, anticaries agents (e.g., xylitol), calcium sources, phosphorus sources, remineralizing agents (e.g., calcium phosphate compounds), enzymes, breath fresheners, anesthetics, clotting agents, acid neutralizers, chemotherapeutic agents, immune response modifiers, thixotropes, polyols, anti-inflammatory agents, antimicrobial agents (in addition to the antimicrobial lipid component), antifungal agents, agents for treating xerostomia, desensitizers, and the like, of the type often used in compositions. Combination of any of the above additives may also be employed. The selection and amount of any one such additive can be selected by one of skill in the art to accomplish the desired result without undue experimentation.

### Packaged Articles, Kits, and Methods

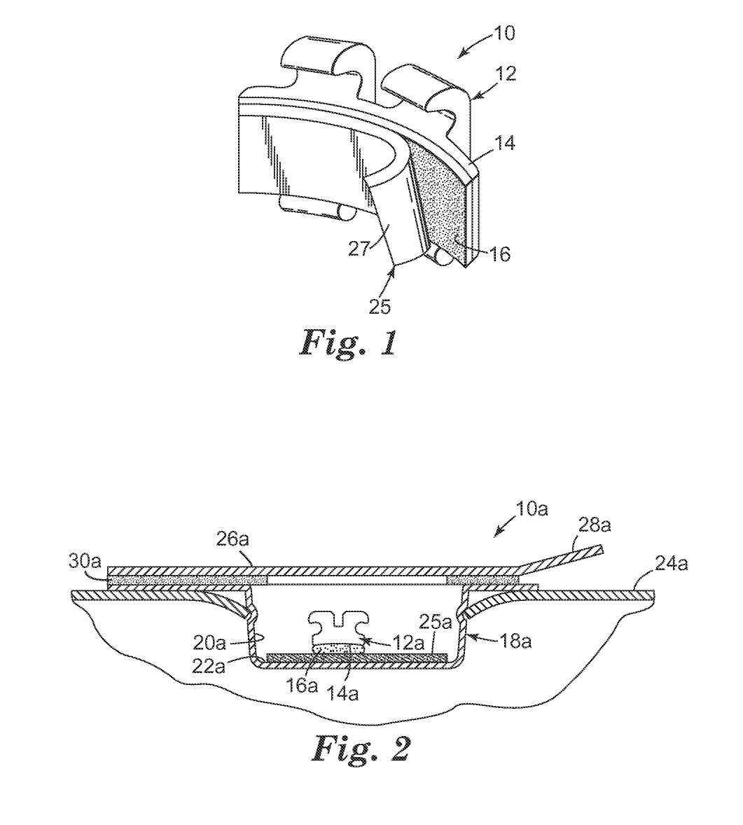

In further embodiments, packaged appliances and kits according to the present invention include an orthodontic appliance coated with a composition of the present invention. One such exemplary embodiment is illustrated in FIG. 1. In FIG. 1, exemplary article 10 includes orthodontic appliance 12 having a base 14 for bonding to a tooth structure. Exemplary appliance 12 here can represent one of a variety of orthodontic appliances including orthodontic brackets, buccal tubes, lingual buttons, lingual sheaths, cleats, and orthodontic bands. These appliances may be made of metal, plastic, ceramic and combinations thereof Preferably the bottom surface of base 14 has a concave compound contour that matches the convex compound contours of the patient's tooth surface (not shown). Optionally the bottom surface of base 14 is provided with grooves, particles, recesses, undercuts, a chemical bond enhancement material, or any other material or structure or combination thereof that facilitates bonding the appliance 12 directly to a patient's tooth. Article 10 further includes a composition 16 in contact with base 14. Details concerning the characteristics of the composition 16 have already been described in detail and will not be repeated here.

It should be understood that article 10 can optionally include one or more additional layers of compositions (e.g., orthodontic adhesives, orthodontic primers, or combinations thereof, which are not illustrated in FIG. 1) in contact with base 14 and/or composition 16. Specifically, such additional layer(s) can be between base 14 and composition 16; on composition 16 opposite base 14, or both. Such layers may or may not cover the same area, and may independently be discontinuous (e.g., a patterned layer) or continuous (e.g., non-patterned) materials extending across all or a portion of base 14.

Preferably and as shown, the articlee 10 also includes a release substrate 25 including a surface 27 that is in contact with the composition 16. The release substrate 25 may be selected from a number of materials including, for example, polyolefins, poly(vinyl chloride), polyurethanes, and poly(tetrafluoroethylene). Optionally, the surface 27 of the release substrate 25 comprises a number of pores, and preferably no more than 50% by weight of the composition 16 is within the pores. In certain embodiments, the release substrate 25 includes closed-cell foam materials as disclosed, for example, in U.S. Pat. No. 6,183,249 (Brennan et al.).

The article 10 is preferably packaged in a container that provides barriers to the transmission of light and/or water vapor. In some embodiments of the present invention, the articlee 10 is preferably provided as a kit. In other embodiments, the present invention preferably provides a method of bonding an orthodontic appliance 12 to a tooth, in which the composition 16 includes one or more fillers of the type described.

The compositions of the present invention can also be adapted for indirect bonding methods. For indirect bonding methods, orthodontic appliances are typically placed, for example, on a replica model (such as one made from orthodontic stone or cured epoxy) of the patient's dental arch to provide a custom base for later mounting on the patient's tooth structure, commonly using a placement device or transfer tray. In one embodiment, the orthodontic appliances have a composition coated on their respective bases thereon for bonding to the replica model. Thus, the composition can be seated against the replica model to form a custom base, for example, upon hardening of the composition. Exemplary indirect bonding methods are described in greater detail in U.S. Pat. No. 7,137,812 (Cleary et al.).

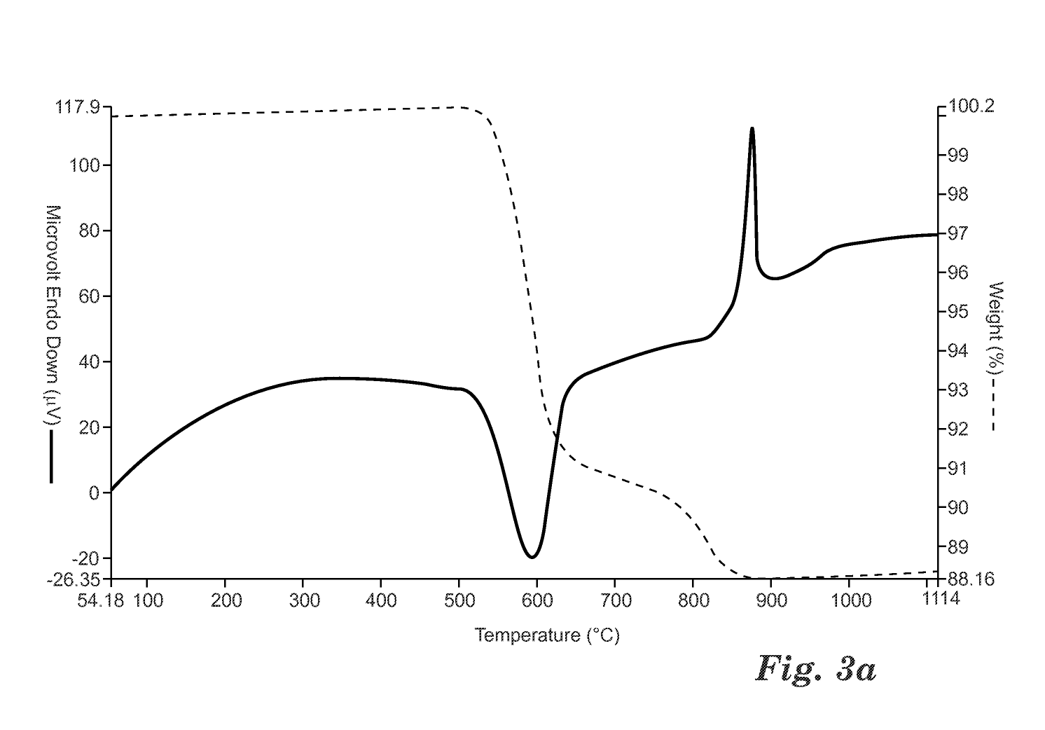

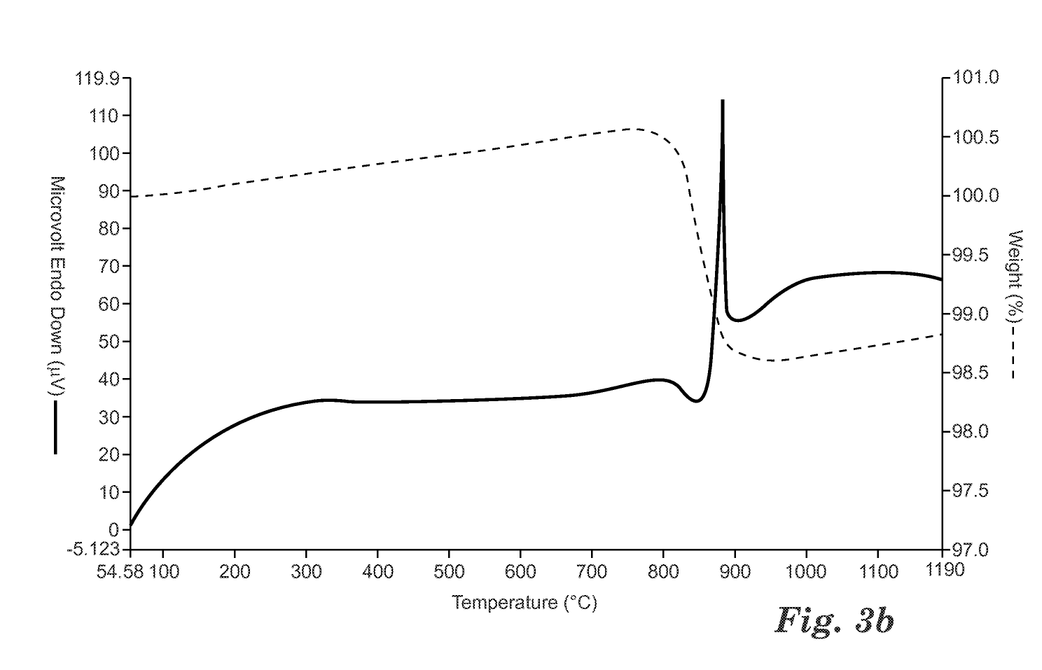

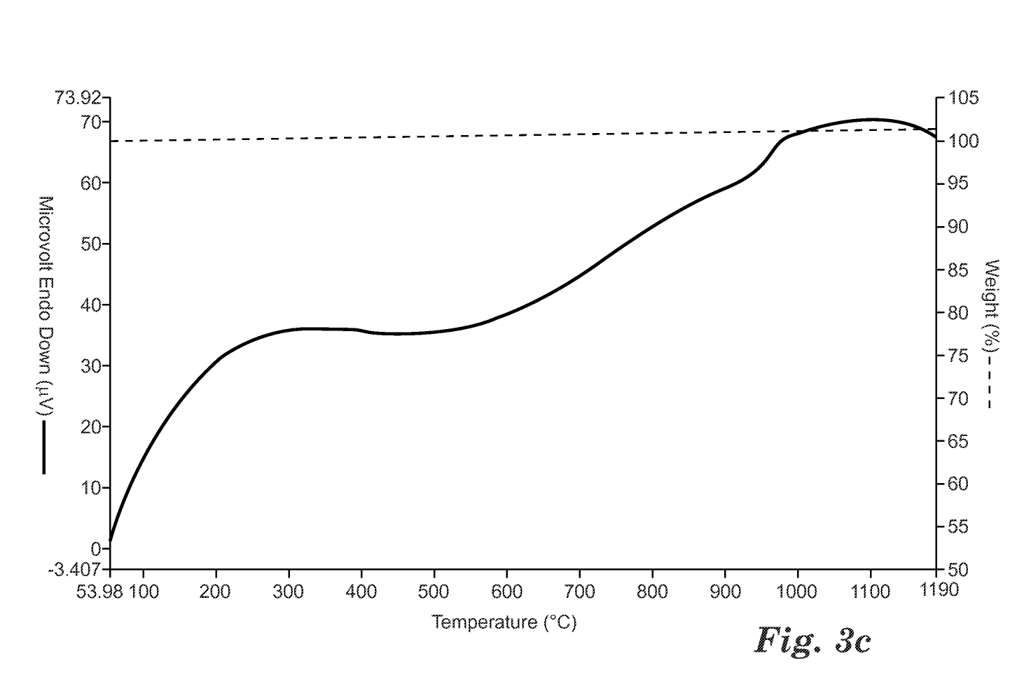

In another embodiment, referring to FIG. 2, the present invention provides a packaged article 10a including a orthodontic appliance 12a. The appliance 12a has a base 14a for directly bonding the appliance 12a to a patient tooth structure (not shown). A composition 16a extends across the base 14a of the bracket 12a. The bracket 12a and the composition 16a are at least partially surrounded by a container 18a. The container 18a illustrated in FIG. 3 includes an integrally-molded body with internal wall portions that define a recess or well 20a. The well 20a includes side walls and a bottom 22a. As an additional option, the side walls of the well 20a include horizontally extending recesses for engagement with edge structure of carrier 24a. Additional information regarding a suitable carrier 24a is set out in U.S. Pat. No. 5,328,363 (Chester et al.). Preferably, the bottom 22a of the well 20a includes a release substrate 25a. Preferably, the container 18a also includes a cover 26a with a tab 28a, with the cover 26a being releasably connected to the container 18a by, for example, adhesive 30a.

In preferred embodiments, the container 18a provides excellent protection against degradation of the composition(s) (e.g., photopolymerizable compounds), even after extended periods of time. Such containers 18a are particularly useful for embodiments in which the composition includes dyes that impart a color changing feature to the composition, as described previously. It is further preferable that containers 18a effectively block the passage of actinic radiation over a broad spectral range, and as a result, the composition does not prematurely lose color during storage.

In preferred embodiments, the container 18a comprises a polymer and metallic particles. As an example, the container 18a may be made of polypropylene that is compounded with aluminum filler or receives an aluminum powder coating as disclosed, for example, in U.S. Patent Publication No. 2003/0196914 A1 (Tzou et al.). The combination of polymer and metallic particles is highly effective in blocking the passage of actinic radiation to color changing dyes, even though such dyes are known to be highly sensitive to light. Such containers also exhibit good vapor barrier properties. As a result, the rheological characteristics of the composition(s) are less likely to change over extended periods of time. For example, the improved vapor barrier properties of such containers provide substantial protection against degradation of the handling characteristics of orthodontic compositions so that they do not prematurely cure or dry or become otherwise unsatisfactory. Suitable covers 26a for such containers can be made of any material that is substantially opaque to the transmission of actinic radiation so that the compositions therein do not prematurely cure. Examples of suitable materials for cover 26a include laminates of aluminum foil and polymers. For example, the laminate may comprise a layer of polyethyleneterephthalate, adhesive, aluminum foil, adhesive and oriented polypropylene.

In some embodiments, a packaged article can include a set of two or more orthodontic appliances, wherein at least one of the appliances has an orthodontic composition thereon. Additional examples of appliances and sets of appliances are described in U.S. Patent Application Publication No. 2005/0133384 A1 (Cinader et al.). Packaged orthodontic appliances are described, for example, in U.S. Patent Application Publication No. 2003/0196914 A1 (Tzou et al.) and U.S. Pat. No. 4,978,007 (Jacobs et al.), U.S. Pat. No. 5,015,180 (Randklev), U.S. Pat. No. 5,328,363 (Chester et al.), and U.S. Pat. No. 6,183,249 (Brennan et al.).

Still another embodiment provides a method for removing the hardened composition from the surface of a tooth. In this embodiment, a tooth surface is provided and a hardened composition of the present invention resides on at least a portion of the tooth surface thereon. The term “tooth” in this case may represent not only a patient's actual tooth but also a physical replica of a patient's tooth such one provided by an orthodontic stone or cured epoxy model. The method further includes applying an abrasive to the tooth surface to remove the cured composition from the tooth, wherein the abrasive has a Mohs hardness that is less than 5. In certain embodiments, the abrasive has a Mohs hardness that is less than 4.5, or less than 4, or less than 3.5.

This abrasive can take the form of an abrasive particle that is coated on a substrate, such as a finishing disk or gritted sandpaper. One example of a suitable coated abrasive is a SOF-LEX finishing disks from 3M Company in St. Paul, Minn. For a finishing disk, a rotation speed of 10,000 rpm can be used. As an alternative, it is possible to use prophylactic (or prophy) treatment, which is commonly used to clean the teeth of a patient prior to bonding. Prophy treatment may include pumice powders such as those available from dental distributors. Fine, medium or coarse grain pumice (prophy paste), along with a prophy cup and prophy angle, can be used. A finishing burr or an edged dental hand instrument, such as a scalar, could also be used.

Additional aspects of this invention are further illustrated by the following examples. Particular materials and amounts thereof recited in these examples, as well as other conditions and details, however, should not be construed to unduly limit this invention. Unless otherwise indicated, all parts and percentages are on a weight basis, all water is deionized water, and all molecular weights are weight average molecular weight. Unless otherwise noted, all reagents were obtained from Sigma-Aldrich Corp. in St. Louis, Mo.

### EXAMPLES

As used herein,

“BisGMA” refers to 2,2-bis[4-(2-hydroxy-3-methacryloyloxypropoxy)phenyl]propane;

“BHT” refers to 2,6-Di-tert-butyl-4-methylphenol;

“CM-111” refers to a flame-fused talc filler, provided by 3M Company;

“CPQ” refers to camphorquinone;

“EDMAB” refers to ethyl-4-(N,N-dimethylamino)benzoate;

“Iodonium PF₆” refers to iodonium hexafluorophosphate;

“HEMA” refers to 2-hydroxyethyl methacrylate, provided by 3M Company;

“TEGDMA refers to tetraethylene glycol dimethacrylate, provided by 3M Company;

“UDMA” refers to urethane dimethacrylate, provided by 3M Company;

“Sierralite 402HS” refers to chlorite from Luzenac (Greenwood Village, Colo.);

“S/T” denotes that the material has been silane treated;

“Schott Glass” refers to fluoroaluminosilicate glass prepared and described in

Preparatory Example 1 in International Patent Application Publication No. WO 00/69393 (Brennan et al.);

Below is a description of various experimental procedures to be cited in the Examples:

### Calcining Process

100 g of the hydrated mineral of interest was spread evenly in a 6″×3.5″ fused silica tray provided by Technical Glass Products, Inc (Painesville Twp., Ohio) and heated at 6 C./min to 950° C. in a F48025-80 Thermolyne Type 4800 furnace provided by Barnstead International (Dubuque, Iowa). The mineral was allowed to sit at 950° C. for the desired length of time (typically two hours) at which point the furnace was allowed to cool naturally to room temperature. The tray was removed from the room temperature furnace and the heat treated mineral was reweighed and the loss on ignition determined. Loss on Ignition=(Initial Mass (100 g)−Mass After Heating)/Initial Mass (100 g)

### Silane Treatment

Silane treatment took place by suspending the particles in a 50/50 (v/v) mixture of IPA and water. A suitable amount of A-174 Silane (from Alfa Aesar in Ward Hill, Mass.) was then added, typically in an amount ranging from 1 to 5 weight percent of the total filler amount. Unless otherwise noted, all heat-modified fillers used in these examples were silane treated using a 1 percent solution of A-174 Silane in water. The pH was then adjusted to ˜9 and stirred overnight at RT. The material is then tray dried at 85° C. for 6 hours and 100° C. for 2 hours. The material is then screened though a 74 micron mesh to filter out agglomerates and used as is.

### Filler-Resin Compounding

The handling of all photosensitive materials took place under yellow lights. The CPQ, EDMAB, and Iodonium PF₆⁻ were dissolved in their respective resins by alternately heating the mixture in a 65° C. oven and subsequent mechanical mixing until no solids were visible. Preparation of the composite pastes was carried out using a DAC-150FZ SpeedMixer (Flaktec Corp. Landrum, S.C.). Each batch was mixed at a rotational speed of 3000 rpm for three 1-minute time intervals. Care was taken not to exceed the maximum filler loading that would provide a stable and uniform composite paste.

### Shear Bond Strength Test

All shear bond strength measurements were conducted on uncut bovine teeth, which were cleaned and partially embedded in circular polymethylmethacrylate discs with the labial tooth surface exposed. All teeth underwent further prophy treatment for 30 seconds using ORAL-B brand medium-sized pumice powder (from Patterson Companies, Inc. in St. Paul, Minn.), rinsing with water and drying using an air syringe immediately prior to bonding. The dry enamel was then etched and primed by rubbing TRANSBOND PLUS brand Self Etching Primer (from 3M Unitek in Monrovia, Calif.) on each tooth for 3 seconds according to the instructions for use for this primer. A gentle air burst was used to thinly spread and dry the primer on the tooth surface to be bonded.

To prepare each test specimen, approximately 10 mg of test composition was applied to the base of a VICTORY SERIES brand upper left central bracket (part no. 017-401, from 3M Unitek), and the coated bracket firmly seated onto the tooth surface. Excess composition expressed around the periphery of the bracket base was subsequently removed using a 0/1 Marquette Condenser (part no. PLG 0/1, from Hu-Friedy in Chicago, Ill.), taking care not to inadvertently disturb the bracket position. The composition was then photocured by exposure to actinic radiation using a 3M ESPE ELIPAR brand TRILIGHT curing light unit (from 3M ESPE in St. Paul, Minn.) for 10 seconds on two opposite sides of the bracket. The above process was repeated for as many bonding test specimens as needed to obtain a complete set of replicated samples. After all specimens were fully bonded, they were submerged in water maintained at 37° C. for 24 hours.

Debonding was conducted on each test specimen using an Insight 5 test instrument (from MTS in Eden Prairie, Minn.) outfitted with a 500 N load cell. For each debonding, the test specimen was mounted in a fixture, then a 0.44 mm (0.017 inches) diameter stainless steel wire fixed to a crosshead was looped beneath the occlusal tiewings of the bracket and the crosshead was translated upwards at 5.1 mm (0.20 inches) per minute until shear failure was observed. Raw force data were converted to force per unit area (units of kg/cm² or megapascals) using the known bracket base area (10.6 mm², or 0.0164 inches², for the VICTORY SERIES brand upper left central brackets used).

The adhesive remnant index (ARI) was also evaluated for each composition upon debonding. The ratings depict the remaining amount of adhesive left on the bracket as follows:

0, no adhesive left on the tooth

1, less than half of adhesive left on the tooth

2, more than half of the adhesive left on the tooth

3, all of the adhesive left on the tooth, with distinct impression of the bracket mesh

To maintain consistency during testing, all compositions within a series were conducted side-by-side by a single operator, and ambient temperature and humidity were held as constant as possible throughout the test. For each composition tested, the mean and standard deviation of shear bond strength and mean ARI were reported for a set of at least 5 replicated test measurements.

### Thermal Gravimetric-Differential Thermal Analysis

Thermal gravimetric-differential thermal analysis was performed on filler samples both prior to and after heat-modification to determine changes in weight in relation to changes in temperature. In these examples, the sample to be tested is typically trimmed to a weight ranging from 15-30 mg and then placed in a tiny platinum pan. The pan is then loaded into a semi-automated PYRIS DIAMOND brand thermal gravimetric-differential thermal analyzer (TG-DTA) (from Perkin Elmer in Waltham, Mass.) and purged with air to aid in removal of products of ignition. Analysis was carried out by first holding the sample steady at 50 degrees Celsius for 1 minute, then applying heat to raise the temperature from 50 degrees to 1170 degrees at a ramp rate of 10 degrees per minute. Once temperature reached 1170 degrees Celsius, the test was terminated and the sample allowed to return back to ambient temperature. During the course of this thermal cycle, both heat flow as well as sample weight were monitored and plotted as a function of temperature.

### Diametral Tensile Strength Testing

In preparation, the composition sample to be tested is place in a glass tube with an ID of 4 mm, placed under 35 PSI of pressure to remove any air bubbles and cured for 60 seconds with a halogen curing light. The cured sample is then cut into ˜2 mm long cylinders using a diamond saw and stored in water @ 37° C. for 24 hours before testing. The samples are then dried, the diameter and length measured and placed between two steel compressive plates. The sampled is then stressed at the rate of 0.05 in/min until fracture occurs. The diametral tensile strength can then be calculated from the recorded force by using the formula:

Tensile (in PSI)=(lbs. force)(2)/(L)(D)(3.1416)

### EXAMPLES

### Examples 1-2 and Comparative Examples CE-1, CE-2

Examples 1 and 2 demonstrate the shear bond strength performance of compositions containing heat-modified talc fillers compared to a control composition containing virgin talc filler as well as a commercially available control. In this comparison, Example 1 uses a talc powder (99% pure 2.4 micron diameter) that has been calcined according to the procedure described earlier (“Calcining Process”), while Example 2 uses CM-111, a flame-fused talc filler. Comparative Example CE-1 uses the virgin talc powder, while CE-2 represents TRANSBOND XT (TBXT) brand orthodontic adhesive. It is noted that TBXT contains a filler derived from quartz, which is substantially harder than talc. All talc fillers were silane treated prior to use as described earlier (see “Silane Treatment”). The composite pastes were then prepared and bond strength measurements conducted according to the procedures described earlier (see “Filler-Resin Compounding” and “Shear Bond Strength Test”).

The formulations of the compositions tested, along with the corresponding mean and standard deviation for shear bond strength, and mean adhesive remnant index (ARI), are listed in Table 1. During preparation, it was noted that both the virgin talc (CE-1) and calcined talc (Example 1) displayed a maximum filler loading of only about 60 weight percent, while the flame fused talc (Example 2) displayed a maximum filler loading of about 70 percent. The shear bond strength data show that both calcined and flame fused talc filler provide adhesive compositions superior to that of the non-modified talc filler control. The data further suggest that the flame fused filler in Example 2 supports bond strengths comparable to that of the benchmark Transbond XT adhesive.

These results are reinforced by the p-values given in Table 2 below, which indicate the degree of statistical significance in observed bond strength differences. In this disclosure herein, p-values less than or equal to 0.05 are considered indicative of a statistically significant difference. Based on this criterion, both Example 1 and Example 2 show a statistically significant improvement in bond strength over CE-1. It is further noted that Example 2 shows a bond strength statistically equivalent to that of CE-2 (TBXT). The ARI data for the flame-fused filler systems averaged 3.0, indicating bond failure at the interface between the bracket base and the composition. By contrast, ARI data for the control compositions, CE-1 and CE-2, indicate cohesive failure through the composition itself

TABLE 1
Compositions and shear bond strength data for Examples
1-2 and Comparative Examples CE-1, CE-2

| | | | | CE-2 |
|---|---|---|---|---|
| Component | 1 | 2 | CE-1 | (TBXT) |
| UDMA | 41.08% | 28.38% | 39.08% | |
| CPQ | 0.21% | 0.14% | 0.20% | |
| EDMAB | 0.42% | 0.29% | 0.40% | |
| Iodonium PF₆⁻ | 0.21% | 0.14% | 0.20% | |
| BHT | 0.04% | 0.03% | 0.04% | |
| 1% S/T virgin talc powder | 0 | 0 | 60.08% | |
| 1% S/T calcined | 58.04% | 0 | 0 | |
| virgin talc powder | | | | |
| 1% S/T CM-111 | 0 | 71.01% | 0 | |
| Shear bond strength, | 13.8 | 18.1 | 10.8 | 19.2 |
| mean (MPa) | | | | |
| Shear bond strength, standard | 3.0 | 4.8 | 2.1 | 5.4 |
| deviation (MPa) | | | | |
| Adhesive Remnant Index, mean | 3.0 | 3.0 | 2.2 | 2.5 |

TABLE 2
Calculated p-values between shear bond strength data in Table 1.

| | Example 1 | Example 2 | CE-1 | CE-2 |
|---|---|---|---|---|
| Example 1 | | 0.017 | 0.010 | 0.008 |
| Example 2 | 0.017 | | 0.000 | 0.601 |
| CE-1 | 0.010 | 0.000 | | 0.000 |
| CE-2 | 0.008 | 0.601 | 0.000 | |

### Examples 3-4 and Comparative Examples CE-3, CE-4

In a second series of measurements, the effects of using a calcined chlorite filler were examined. The chlorite filler, Sierralite 402HS, was calcined in house and used to prepare the composition in Example 3. Virgin (uncalcined) Sierralite 402HS filler was used as received to prepare the composition of Comparative Example CE-3. Two additional samples were included in this comparison—Example 4, which used the CM-111 flame-fused talc as filler, and Comparative Example CE-4 (TBXT). The shear bond strength and adhesive remnant index data are presented in Table 4 below.

The shear bond strength data again demonstrate that the compositions containing calcined or flame-fused filler display higher adhesive strength compared to those containing untreated chlorite filler. Furthermore, Examples 3 and 4 show bond strength values comparable to that of the benchmark CE-4 (TBXT). The ARI data for Examples 3 and 4 are consistent with a mixture of both cohesive and adhesive failure. The p-values for this series of data are given in Table 4, and also demonstrate that there is a statistically significant difference between compositions containing the calcined chlorite versus the virgin chlorite. Examples 3 and 4, on the other hand, yielded statistically similar bond strengths to that of CE-4.

TABLE 3
Compositions and shear bond strength data for Examples
3-4 and Comparative Examples CE-3, CE-4

| | | | | CE-4 |
|---|---|---|---|---|
| Component | 3 | 4 | CE-3 | (TBXT) |
| UDMA | 41.13% | 28.39% | 38.31% | |
| CPQ | 0.21% | 0.15% | 0.20% | |
| EDMAB | 0.42% | 0.29% | 0.39% | |
| Iodonium PF₆⁻ | 0.21% | 0.15% | 0.20% | |
| BHT | 0.04% | 0.03% | 0.04% | |
| 1% S/T Calcined 402HS | 57.99% | 0 | 0 | |
| Chlorite | | | | |
| 1% S/T CM-111 Talc | 0 | 71.00% | 0 | |
| 1% S/T 402HS Chlorite | 0 | 0 | 60.87% | |
| Shear bond strength, | 18.3 | 18.0 | 14.6 | 20.6 |
| mean (MPa) | | | | |
| Shear bond strength, standard | 3.7 | 2.4 | 3.0 | 4.2 |
| deviation (MPa) | | | | |
| Adhesive Remnant Index, mean | 2.6 | 2.8 | 3.0 | 2.3 |

TABLE 4
Calculated p-values between shear bond strength data in Table 3.

| | Example 3 | Example 4 | CE-3 | CE-4 |
|---|---|---|---|---|
| Example 3 | | 0.804 | 0.010 | 0.182 |
| Example 4 | 0.804 | | 0.007 | 0.086 |
| CE-3 | 0.010 | 0.007 | | 0.001 |
| CE-4 | 0.182 | 0.086 | 0.001 | |

### Examples 5-6 and Comparative Examples CE-5, CE-6

Examples 5 and 6 demonstrate the diametral tensile strength of compositions using both calcined and virgin chlorite fillers. Example 5 shows an composition containing the silane-treated Sierralite filler after calcination. Example 6 shows a similar composition, differing from Example 5 in that it includes about 8 weight percent BisGMA resin. Comparative Examples CE-5 and CE-6 use identical formulations to Examples 5 and 6, with the exception that the calcined Sierralite filler was replaced with virgin Sierralite filler. The compositions and diametral tensile strength for Examples 5-6 and CE-5, CE-6 are listed in Table 5 below.

TABLE 5
Compositions and diametral tensile strength data for
Examples 5-6 and Comparative Examples CE-5, CE-6

| Component | 5 | 6 | CE-5 | CE-6 |
|---|---|---|---|---|
| UDMA | 40.14% | 33.68% | 38.18% | 32.10% |
| BisGMA | 0 | 8.42% | 0 | 8.03% |
| CPQ | 0.21% | 0.22% | 0.20% | 0.20% |
| EDMAB | 0.41% | 0.43% | 0.39% | 0.41% |
| Iodonium PF₆⁻ | 0.21% | 0.22% | 0.20% | 0.20% |
| BHT | 0.04% | 0.04% | 0.04% | 0.04% |
| 1% S/T Calcined 402HS | 59.00% | 57.00% | 0 | 0 |
| Chlorite | | | | |
| 1% S/T 402HS Chlorite | 0 | 0 | 61.00% | 59.01% |
| Diametral tensile strength, | 33.554 | 33.347 | 27.416 | 26.722 |
| mean (MPa) | | | | |
| Diametral tensile strength, | 3.036 | 6.029 | 1.505 | 0.586 |
| standard deviation (MPa) | | | | |

These results show that calcining the chlorite filler results in a significant improvement in diametral tensile strength. This observation holds true for both formulations containing and not containing BisGMA. The statistical significance attributed to calcining was further verified by the p-values obtained between Example 5 and CE-5 (P=0.003) and Example 6 and CE-6 (P=0.044). Examples 5 and 6 also suggest that BisGMA resin blends can be used to modify handling properties without significantly impacting diametral tensile strength.

### Example 7 and Comparative Examples CE-7

Example 7 demonstrates the use of flame-fused talc in a hybrid filler system that includes both CM-111 as well as a fluoride ionomer glass. Unlike previous formulations which were silane treated at 1% concentration, the talc filler in this example was silane treated at a 5% concentration. The formulation of this composition, and its bond strength performance as compared to Comparative Example CE-7 (TBXT) is shown in Table 6 below. The mean ARI values were also listed in Table 6.

TABLE 6
Compositions and shear bond strength data
for Example 7 and Comparative Examples CE-7

| | | CE-7 |
|---|---|---|
| Component | 7 | (TBXT) |
| UDMA | 22.38% | |
| CPQ | 0.11% | |
| EDMAB | 0.23% | |
| Iodonium PF₆⁻ | 0.11% | |
| BHT | 0.02% | |
| 5% S/T CM-111 Talc | 38.62% | |
| FAS Glass | 38.52% | |
| Shear bond strength, | 21.9 | 19.4 |
| mean (MPa) | | |
| Shear bond strength, standard | 3.7 | 3.5 |
| deviation (MPa) | | |
| Adhesive Remnant Index, mean | 2.8 | 2.6 |

Example 7 displayed a mean shear bond strength similar to that of the comparative example CE-7 (P=0.112). This result shows that the inclusion of FAS glass did not adversely affect the shear bond strength of the resulting orthodontic composition. The ARI values for both compositions were consistent with a mixture of both cohesive and adhesive failure upon debonding.

### Example 8 and Comparative Examples CE-8

To test for the rate of removal, cured cylinders of the indicated composition (4 mm in diameter) were abraded with a Sof-Lex Medium Coarse disk at 20,000 rpm for 15 seconds and 20 grams of normal force applied against the abrasive. In this test, shorter times for remnant removal is better. Table 7 below shows both the measured rate of mass removal and the projected time to abrade away a 10 mg slug of hardened composition. The comparison was made between two compositions: Example 8 represents a cured UDMA-based composition containing flame-fused CM-111 talc filler, and Comparative Example CE-8 represents cured Transbond XT adhesive. As shown, Example 8 displayed a removal rate that was about an order of magnitude faster than that of the control, CE-8.

TABLE 7
Compositions and abrasion test data for
Example 8 and Comparative Examples CE-8

| | | CE-8 |
|---|---|---|
| Component | 8 | (TBXT) |
| UDMA | 28.39% | |
| CPQ | 0.15% | |
| EDMAB | 0.29% | |
| Iodonium PF₆⁻ | 0.15% | |
| BHT | 0.03% | |
| 1% S/T CM-111 Talc | 71.00% | |
| Rate of mass removal, | 0.212 | 0.019 |
| mean (mg/s) | | |
| Rate of mass removal, | 0.127 | 0.009 |
| standard deviation (mg/s) | | |
| Expected time | 47 | 517 |
| to remove 10 mg (s) | | |

### Examples 9-11 and Comparative Examples CE-9, CE-10

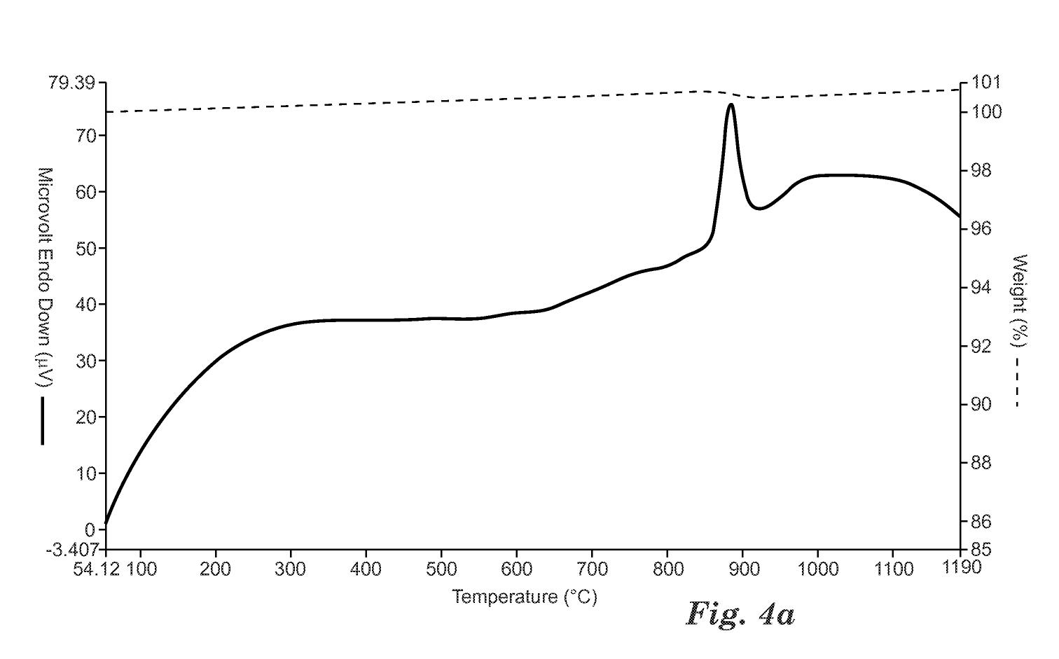

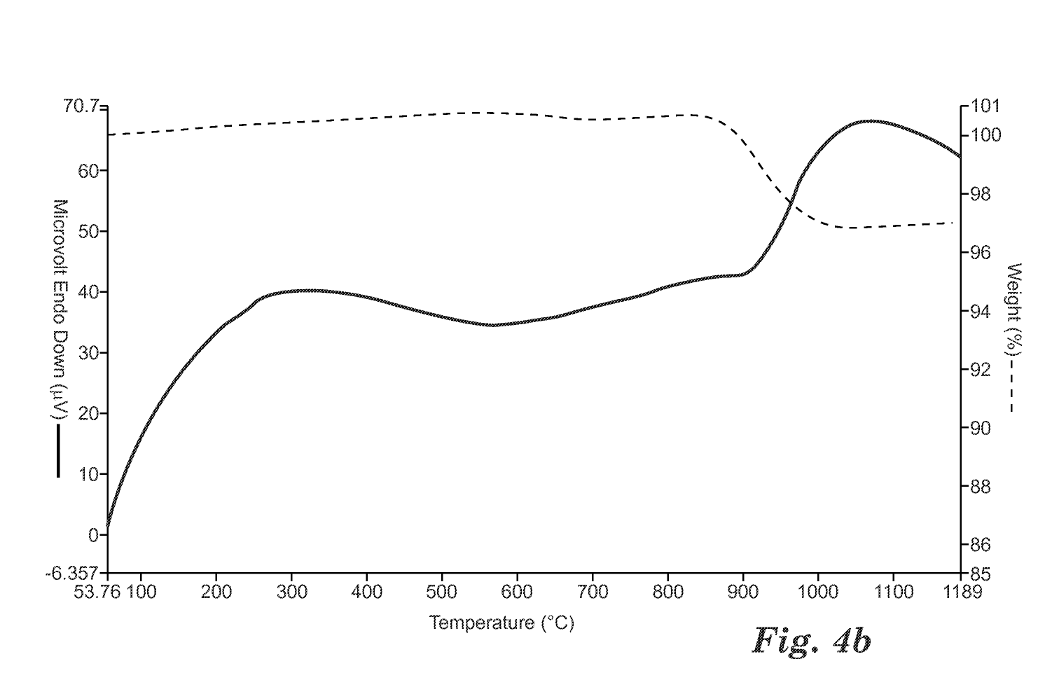

To better understand the differences between the virgin and heat modified filler materials tested, thermal gravimetric-differential thermal analysis (TG-DTA) was conducted on exemplary chlorite and talc filler materials, before and after heat modification. Details on the TG-DTA method were described previously (see “Thermal Gravimetric-Differential Thermal Analysis”). Testing on the chlorite fillers included three Sierralite 402HS fillers: in the virgin state (Comparative Example CE-9), calcined at 800 degrees Celsius (Example 9), and calcined at 950 degrees Celsius (Example 10). The talc fillers tested included the virgin talc powder (Comparative Example CE-10) and flame fused CM-111 filler (Example 11). All fillers were silane treated as previously described. The TG-DTA traces corresponding to CE-9 and Examples 9-10 are shown in FIGS. 3a, 3b, and 3c. The traces corresponding to CE-10 and Example 11 are shown in FIGS. 4a and 4b. Peak locations and respective percent weight loss data are provided in Table 8 below.

TABLE 8
TG-DTA peak locations and weight loss data for chlorite filler
materials in the virgin state and after calcination

| Filler | Filler, | Peak 1 | Wt. loss | Peak 2 | Wt. loss |
|---|---|---|---|---|---|
| Example | Treatment | (° C.) | (%) | (° C.) | (%) |
| CE-9 | S/T 402HS, | 595 | 9 | 876 | 1.5 |
| | virgin | | | | |
| 9 | S/T 402HS, | not | N/A | 882 | 1.9 |
| | calcined at 800° C. | present | | | |
| 10 | S/T 402HS, | not | N/A | not | N/A |
| | calcined at 950° C. | present | | present | |
| CE-10 | S/T virgin talc powder, | not | N/A | 885 | 3.8 |
| | virgin | present | | | |
| 11 | S/T CM-111, | not | N/A | not | N/A |
| | flame fused | present | | present | |

The TG-DTA trace for the virgin chlorite filler materials in FIG. 3a and the data in Table 8 reveal an endothermic transition near 590° C. and exothermic transition near 875° C.; these are identified here as ‘Peak 1’ and ‘Peak 2’, respectively. Both peaks are absent in Examples 10 (FIG. 3c) while Example 9 (FIG. 3b) shows the exothermic peak but not the endothermic peak. It is further noted that the endothermic transition was accompanied by a measured weight loss of about 9 weight percent suggesting the presence of a hydrated chlorite phase. The disappearance of the endothermic peak in Examples 9 and 10 is consistent with the elimination of the hydrated phase as a result of calcining By contrast, the exothermic peak observed in CE-9 and Example 9, but absent from Example 10, was accompanied by a much smaller weight loss ranging from 0.5 to 1.5 weight percent. It is presumed that this peak is indicative of some other high temperature phase transition. In summary, the TG-DTA data demonstrates that a hydrated phase exists in virgin chlorite filler, that this hydrated phase may be eliminated through calcining, and that an additional exothermic phase transition occurs at temperatures around 875° C.

The talc fillers also show evidence of a high temperature phase transition as a result of heat modification. As shown by the TG-DTA traces for CE-10 (FIG. 4a), the virgin talc filler displays an exothermic ‘Peak2′ transition near 885° C. The weight loss corresponding to this transition was measured to be about 3.8 weight percent. This transition disappeared after the filler was flame fused, as shown by Example 11 (FIG. 4b).

The complete disclosure of all patents, patent applications, and publications cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims that follow.

1. An orthodontic composition comprising:
a hardenable component;
a hardener; and
a heat-modified inorganic mineral filler having a Mohs hardness not exceeding 5, wherein some or all water of hydration has been irreversibly eliminated from the inorganic mineral filler to form a non-hydrated phase.

2. The composition of claim 1, wherein less than 5 weight percent of the inorganic mineral filler is hydrated based on the overall weight of the filler.

3. The composition of claim 1, wherein essentially none of the inorganic mineral filler is hydrated.

4. (canceled)

5. The composition of claim 1, wherein the inorganic mineral filler is selected from the group consisting of talc, kaolin, barite, aragonite, calcite and chlorite.

6. The composition of claim 1, wherein the inorganic mineral filler is selected from the group consisting of: flame fused inorganic mineral filler, atomized inorganic mineral filler, fire polished inorganic mineral filler, and directly fused inorganic mineral filler.

7. The composition of claim 1, wherein the inorganic mineral filler is a calcined inorganic mineral filler.

8. The composition of claim 1, wherein the inorganic mineral filler has a mean particle diameter ranging from 1 to 25 micrometers.

9. The composition of claim 1, wherein the inorganic mineral filler has a mean particle diameter ranging from 2 to 20 micrometers.

10. The composition of claim 1, wherein the inorganic mineral filler has a mean particle diameter ranging from 4 to 15 micrometers.

11. The composition of claim 1, wherein the inorganic mineral filler is present in an amount ranging from 50 to 75 percent by weight based on the total weight of the composition.

12. The composition of claim 1, wherein the inorganic mineral filler is present in an amount ranging from 66 to 72 percent by weight based on the total weight of the composition.

13. A method of making an orthodontic composition comprising:
providing a hardenable component;
providing a hardener;
providing a virgin inorganic mineral filler that includes a hydrated phase and has a Mohs hardness not exceeding 5;
heating the virgin inorganic mineral filler to a temperature sufficient to transform at least a portion of the hydrated phase to a non-hydrated phase in order to make a heat-modified filler; and
combining the hardenable component, hardener, and heat-modified filler to prepare the orthodontic composition.

14. The method of claim 13 wherein the virgin inorganic mineral filler is heated above its dissociation temperature for eliminating water of hydration.

15. The method of claim 13 wherein the virgin inorganic mineral filler has a loss on ignition ranging from 10 to 15 percent by weight relative to the overall weight of the virgin filler.

16. A packaged article comprising:
an orthodontic appliance having a base for bonding the appliance to a tooth;
a composition extending across the base of the appliance, the composition comprising
a hardenable component,
a hardener, and
a heat-modified inorganic mineral filler having a Mohs hardness not exceeding 5, wherein some or all water of hydration has been irreversibly eliminated from the inorganic mineral filler to form a non-hydrated phase; and
a container at least partially surrounding the orthodontic appliance and the composition.

17. A method for removing a cured orthodontic composition from a tooth comprising:
providing a tooth surface having the cured orthodontic composition on at least a portion thereon, the composition comprising
a hardenable component,
a hardener, and
a heat-modified inorganic filler having a Mohs hardness not exceeding 5, wherein some or all water of hydration has been irreversibly eliminated from the inorganic mineral filler to form a non-hydrated phase; and
applying an abrasive to the tooth surface to remove the composition from the tooth, wherein the abrasive has a Mohs hardness that is less than 5.
